(19) United States
(12) Patent Application Publication
(10) Pub. No.: US 2025/0152710 A1
(43) Pub. Date: May 15, 2025

(54) MODIFIED PRIMARY IMMUNE CELLS FOR INDUCTION OR ENHANCEMENT OF IMMUNOTHERAPY

(71) Applicants: James L. RILEY, Philadelphia, PA (US); The Wistar Institute of Anatomy and Biology, Philadelphia, PA (US); THE TRUSTEES OF THE UNIVERSITY OF PENNSYLVANIA, Philadelphia, PA (US)

(72) Inventors: Luis Montaner, Philadelphia, PA (US); Costin Tomescu, Philadelphia, PA (US); James L. Riley, Philadelphia, PA (US)

(21) Appl. No.: 18/836,188

(22) PCT Filed: Feb. 8, 2023
(86) PCT No.: PCT/US2023/062229

§ 371 Date: Aug. 6, 2024

Related U.S. Application Data
(60) Provisional application No. 63/308,008, filed on Feb. 8, 2022.

(51) Int. Cl. A61K 40/15; A61K 40/33; A61K 40/34; A61K 40/41; A61K 40/46; C12N 5/0783

(52) U.S. Cl. CPC A61K 40/15; A61K 40/33; A61K 40/34; A61K 40/41; A61K 40/46; C12N 5/0646; C12N 2501/2321; C12N 2510/00

(57) ABSTRACT

Provided herein are compositions with an augmented capacity to mediate ADCC. These compositions include chimeric NK cells—called “Nukes” (NK Enhancement Strategy) that express CD64 Fc receptor from an exogenous nucleic acid molecule, the NK cells having antibodies bound thereto. Methods of using these cells for treatment of HIV, cancer, SARS-COV-2, and other diseases are provided.

### STATEMENT OF GOVERNMENT INTEREST

This invention was made with government support under grant No. AI164570 awarded by the National Institutes of Health. The government has certain rights in the invention.

### BACKGROUND OF THE INVENTION

A mechanism of cell-mediated immune defense involves the engagement of antibodies attached to target cells by Fc receptors expressed by leukocytes, which results in target cell killing. This process is referred to antibody-dependent cell-mediated cytotoxicity (ADCC).

Many broadly neutralizing antibodies (bnAbs) targeting the HIV-1 envelope glycoprotein are being assessed in clinical trials as strategies for HIV-1 prevention, treatment, and antiretroviral-free remission. BnAbs can neutralize HIV-1 and target infected cells for elimination.

What is needed are new therapies for treatment of HIV and other diseases.

### SUMMARY OF THE INVENTION

Compositions and methods are described herein that provide effective and useful tools and methods for the treatment of HIV-1 and other diseases such as COVID19 and cancer. Prior art therapies utilizing off-the-shelf NK cell lines have been hampered by the inability to discriminate self, healthy cells from transformed or pathogen-infected cells. See, Dixon et al, Arming of iPSC-Derived NK Cells Expressing a Novel CD64 Fusion Receptor with Therapeutic Antibodies Represents a Novel Off-the-Shelf, Antigen-Targeting Strategy for Cancer, Blood, 138:Supp 1 (p 406) November 2021, which is incorporated herein by reference. Furthermore, they are recognized as non-self by the subject's immune system, and may be cleared from circulation. Therefore, the “off-the-shelf” cell lines have circumvented these issues by providing attenuated immune response.

In one aspect, a composition is provided that includes modified primary human NK cells that express a high affinity CD64 Fc receptor from an exogenous nucleic acid molecule. The modified NK cells have antibodies bound thereto. In certain embodiments, the antibodies are broadly neutralizing anti-HIV-1 antibodies.

In another aspect, a composition is provided that includes modified primary human NK cells that express a high affinity CD64 Fc receptor from an exogenous nucleic acid molecule. The modified NK cells have anti-HIV antibodies bound thereto. In certain embodiments, the anti-HIV antibodies are broadly neutralizing antibodies.

In another aspect, a method of inducing or augmenting antibody-dependent cellular cytotoxicity (ADCC) in a subject in need thereof is provided. The method includes administering a composition that includes modified primary human NK cells that express a high affinity CD64 Fc receptor from an exogenous nucleic acid molecule. The modified NK cells have antibodies bound thereto. In certain embodiments, the antibodies are anti-HIV broadly neutralizing antibodies and the subject is infected with HIV-1.

In another aspect, a method of inducing or augmenting antibody-dependent cellular cytotoxicity (ADCC) in a subject in need thereof is provided. The method includes administering a composition that includes modified primary human NK cells that express high affinity CD64 Fc receptor from an exogenous nucleic acid molecule. The modified NK cells have antibodies bound thereto. In certain embodiments, the antibodies are broadly neutralizing anti-HIV antibodies and the subject is infected with HIV-1.

Other aspects and advantages of these compositions and methods are described further in the following detailed description of the preferred embodiments thereof.

### BRIEF DESCRIPTION OF THE DRAWINGS

FIG. 1A and FIG. 1B show flow cytometric analysis of HIV-specific BNAb Capture and Retention by CD64 Transduced or Control NK cells. Human NK cells were negatively selected from PBMC to high purity using magnetic beads and stimulated with a cocktail of cytokines to trigger NK activation. Cytokine stimulated NK cells were then transduced with a Lentivirus encoding the high-affinity Fc receptor CD64 or mock transduced. Following culture for 11 days, CD64 transduced (FIG. 1A) and Control NK cells (FIG. 1B) were incubated for 60 minutes with 100 mg/mL of a FITC conjugated HIV-specific BNAb 10-1074. CD64 transduced and Control NK cells were then washed twice to remove unbound antibody and incubated for an additional 24 or 48 hours. After each timepoint, CD64 transduced and Control NK cells were stained with antibodies to CD56, CD3, CD16, and CD64. Samples were run on a LSR14 Cytometer and analyzed with FlowJo v10 software. Flow Cytometric analysis of HIV-specific BNAb Capture (10-1074 FITC) is shown on X-axis compared to CD64 expression on Y-axis. Note that Control NK cells could bind the FITC conjugated HIV-specific BNAb 10-1074 weakly at 1 hour but not retain it over time, while CD64 transduced NK cells could bind FITC conjugated HIV-specific BNAb 10-1074 strongly and retain it for several days.

FIG. 2A and FIG. 2B show CD64 Transduced NK cells Can Mediate Strong ADCC against autologous HIV-1 infected CD4+ primary T cells After Pre-loading with HIV-specific BNAbs. Control (FIG. 2A) or CD64 Transduced (FIG. 2B) NK cells were prepared as in FIG. 1 and FACS sorted to high purity to serve as effector cells. PHA/IL-2 stimulated CD4+ primary T cells from the same donor were infected with the IIIB HIV-1 lab isolate for 4 days to serves as autologous target cells. Control or CD64 Transduced NK cells were then incubated with autologous uninfected or HIV-1 infected CD4+ primary T cells a 1:1 effector to target cell ratio in a standard 3-hour degranulation assay with a PE-conjugated anti-CD107a mAb in the presence or absence of HIV-specific BNAbs. To trigger ADCC, 100 μg/mL of the HIV-1 specific BNAbs 10-1074 and 3BNC117 were added either to the NK cells for 15 minutes and washed twice prior to co-culture with target cells or added to targets cells for the entire 3 hour assay. CD107a degranulation was used as a surrogate measure ofNK cell cytotoxicity and was measured on CD56+/CD3− gated NK cells (Dark, blue histogram). Gates were set based upon No Target control NK cells incubated in the absence of targets (Pink, shaded histogram). Note that Control NK cells could only mediate ADCC degranulation against autologous HIV-1 infected CD4+ primary T cells when HIV-1 specific BNAbs added to targets cells for the entire 3 hour assay while CD64 Transduced NK cells could mediate ADCC degranulation against autologous HIV-1 infected CD4+ primary T cells when HIV-1 specific BNAbs added either to targets cells for the entire 3 hour assay as well as to the NK cells for 15 minutes and then washed off.

FIG. 3A and FIG. 3B show ADCC Functional Analysis of CD64 Transduced NK Cells Against HIV-1 Infected Autologous CD4 Primary T Cells. FIG. 3A is a composite graph of Means from 6 individual experiments where BNAb binding by CD64 transduced and Control NK cells was determined at 1 hour post incubation with BNAbs and after washing to remove unbound BNAbs. Statistical analysis of two groups was carried out using a Wilcox on matched-pairs signed rank test with a Two-tailed P-value. FIG. 3B is a composite graph of 6 individual experiments measuring ADCC CD107a Degranulation by CD64 Transduced and Control NK Cells against HIV-1 infected CD4+ primary T cells after HIV-specific BNAb Pre-Loading. Statistical Analysis of two groups was carried out using a Paired T-test assuming Gaussian Distribution with a Two-tailed P-value.

FIG. 4A and FIG. 4B show an ADCC Elimination Assay Measuring the Ability of BNAb Pre-loaded CD64 Transduced NK cells, but not CD8 T cells, to Eliminate HIV-1 Infected Autologous CD4 Primary T Cells. FIG. 4A shows an HIV-1 Elimination Assay where HIV-1 NL4-3 infected CD4+ primary T cells were incubated with no effector cells but in the presence of 100 mg/mL of HIV-specific BNAbs 10-1074 and 3BNC117 or incubated with control autologous NK cells in the absence of antibody (second panel) at a 1:10 Effector to Target cell ratio for 18 hours. Control autologous NK cells (third panel) or Control autologous CD8 T cells (fourth panel) were pre-loaded with 100 mg/mL of HIV-specific BNAbs 10-1074 and 3BNC117 for 15 minutes, washed and then incubated with HIV-1 NL4-3 infected CD4+ primary T cells at a 1:10 Effector to Target cell ratio for 18 hours. As a positive control, 100 mg/mL of HIV-specific BNAbs 10-1074 and 3BNC117 were added to the HIV-1 NL4-3 infected CD4+ primary T cells during the entire 18-hour incubation in the presence of control NK cells. FIG. 4B shows an HIV-1 Elimination Assay where Uninfected CD4+ primary T cells were incubated with no effector cells but in the presence of 100 mg/mL of HIV-specific BNAbs 10-1074 and 3BNC117 or incubated with CD64 Transduced and sorted autologous NK cells in the absence of antibody (second panel) at a 1:10 Effector to Target cell ratio for 18 hours. CD64 Transduced and sorted autologous NK cells (third panel) or CD64 Transduced and sorted autologous CD8 T cells (fourth panel) were pre-loaded with 100 mg/mL of HIV-specific BNAbs 10-1074 and 3BNC117 for 15 minutes, washed and then incubated with HIV-1 NL4-3 infected CD4⁺ primary T cells at a 1:10 Effector to Target cell ratio for 18 hours. As a positive control, 100 mg/mL of HIV-specific BNAbs 10-1074 and 3BNC117 were added to the HIV-1 NL4-3 infected CD4⁺ primary T cells during the entire 18-hour incubation in the presence of CD64 Transduced and sorted autologous NK cells. The percentage of HIV Intracellular p24 capsid positive/CD4 low productively HIV-1 infected target cells remaining after the 18-hour incubation is shown for each condition.

FIG. 5A, FIG. 5B and FIG. 5C shows flow cytometry data showing that control NK cells and CD64 transduced NK cells express the Trail Ligand while HIV-1 infected CD4 T cells also express the Trail Death Receptors DR4 and DR5. The expression of the Trail Death Receptor Ligand was monitored by flow cytometry on cytokine stimulated Control (FIG. 5A) and CD64 Transduced NK cells (FIG. 5B) on day 8 post transduction. Expression of the Trail Death Receptors DR4 and DR5 was monitored by flow cytometry on autologous uninfected and HIV-1 infected CD4⁺ primary T cells on Day 4 post infection with CXCR4-tropic NL4-3 viral isolate (FIG. 5C).

FIG. 6A and FIG. 6B show Proliferation and Durable Expression of CD64 on Transduced NK Cells Over Time. Human NK cells were purified to high purity using magnetic beads and stimulated with 100 U/mL IL-2, 100 ng/mL IL-15 and 100 ng/mL IL-21 cytokines to trigger NK activation. Cytokine stimulated NK cells were CFSE labeled to measure NK cell proliferation then transduced with a Lentivirus encoding the high-affinity Fc receptor CD64. CFSE dilution and CD64 expression was monitored on transduced NK cells by flow cytometry every 3-4 days. Note that CD64 transduced and non-transduced NK cells exhibited equivalent proliferative capacity as evidenced by CFSE dilution. FIG. 6B is a composite graph showing 6 individual transduction experiments where CD64 expression peaked on NK cells around Day 7 and was stably maintained on NK cells over time.

FIG. 7A, FIG. 7B and FIG. 7C shows the maintenance of NK Maturation Over Time on Control and CD64 Transduced NK cells. Human NK cells were purified by negative selection to high purity using magnetic beads and stimulated with 100 U/mL IL-2, 100 ng/mL IL-15 and 100 ng/mL IL-21 cytokines to maintain viability and NK maturation. Expression of the CD57 maturation marker on NK cells was monitored by flow cytometry every 3-4 days on cytokine stimulated control NK cells (FIG. 7A). Following transduction with a Lentivirus encoding the high-affinity Fc receptor CD64, NK cell expression of the CD57 maturation marker was measured on CD64 transduced NK cells as well as control NK cells on Day 7 (FIG. 7B). FIG. 7C is a composite graph showing 7 individual transduction experiments where CD57 expression remained unaltered on transduced NK cells compared to control NK cells at Day 7. Statistical Analysis of two groups was carried out using a Wilcoxon matched-pairs signed rank test with a Two-tailed P-value.

### DETAILED DESCRIPTION OF THE INVENTION

As part of a combined CURE immuno-therapy strategy against HIV, we transduced primary human NK cells with the high affinity CD64 Fc receptor and pre-loaded them with HIV-specific BNAbs. We named these chimeric NK cells “Nukes” (NK Enhancement Strategy) for their augmented capacity to mediate ADCC and their potential clinical application as an autologous primary NK cell immuno-therapy against HIV. As used herein, these, and other similarly modified cells are referred to as “modified cells”, “modified immune cells”, or “modified NK cells”. Compositions and methods that include these modified NK cells are provided herein for the treatment of HIV-1 and other diseases and conditions, as described herein.

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

### Modified Immune Cells

The compositions and methods described herein utilize modified immune cells that express a heterologous Fc receptor. In certain embodiments, the immune cells are natural killer (NK) cells, and the modified cells may be referred to as “Nukes”. NK cells are cytotoxic lymphocytes of the innate immune system that target stressed, infected, and neoplastic cells. Human NK cells mediate ADCC exclusively through the IgG Fc receptor CD16A (FcγRIIIA). There are two allelic variants of CD16A that have either a phenylalanine or valine residue at position 176 (position 158 if amino acid enumeration does not include the signal sequence). The CD16A-176V variant has a higher affinity for IgG, but CD16A-176F is the dominant allele in humans.

CD64 (FcγR1) binds to monomeric IgG with 2-3 orders of magnitude higher affinity than CD16A. CD64 recognizes the same IgG isotypes as CD16A and is expressed by myeloid cells, including monocytes, macrophages, and activated neutrophils, but not NK cells. In certain embodiments, the human high affinity CD64 receptor is used. The sequence of human CD64 is known in the art, and can be found, e.g., at Genbank accession BC156864. The amino acid and nucleic acid sequences are reproduced below. In certain embodiments, a nucleic acid sequence encoding SEQ ID NO: 95, or a sequence sharing at least 90%, 91%, 92%, 93%, 94%, 95%, 96%, 97%, 98%, 99% is utilized. In certain embodiments, the nucleic acid sequence is SEQ ID NO: 96, or a sequence sharing at least 70%, 75%, 80%, 85%, 90%, 95%, or 99% identity with SEQ ID NO: 96 that encodes CD64.

| SEQ ID NO: 95 | |
|---|---|
| MWFLTTLLLWVPVDGQVDTTKAVITLQPPWVSVFQEETVTLHCEVLHLPGSSS | |
| TQWFLNGTATQTSTPSYRITSASVNDSGEYRCQRGLSGRSDPIQLEIHRGWLLL | |
| QVSSRVFMEGEPLALRCHAWKDKLVYNVLYYRNGKAFKFFHWNSNLTILKTN | |
| ISHNGTYHCSGMGKHRYTSAGISQYTVKGLQLPTPVWFHVLFYLAVGIMFLVN | |
| TVLWVTIRKELKRKKKWNLEISLDSGHEKKVISSLQEDRHLEEELKCQEQKEE | |
| QLQEGVHRKEPQGAT | |
| SEQ ID NO: 96 | |
| 1 | gtacaaaaaa gcagaagggc cgtcaaggcc caccatgtgg ttcttgacaa ctctgctcct |
| 61 | ttgggttcca gttgatgggc aagtggacac cacaaaggca gtgatcactt tgcagcctcc |
| 121 | atgggtcagc gtgttccaag aggaaaccgt aaccttgcac tgtgaggtgc tccatctgcc |
| 181 | tgggagcagc tccacacagt ggtttctcaa tggcacagcc actcagacct cgacccccag |
| 241 | ctacagaatc acctctgcca gtgtcaatga cagtggtgaa tacaggtgcc agagaggtct |
| 301 | ctcagggcga agtgacccca tacagctgga aatccacaga ggctggctac tactgcaggt |
| 361 | ctccagcaga gtcttcatgg aaggagaacc tctggccttg aggtgtcatg cgtggaagga |
| 421 | taagctggtg tacaatgtgc tttactatcg aaatggcaaa gcctttaagt ttttccactg |
| 481 | gaattctaac ctcaccattc tgaaaaccaa cataagtcac aatggcacct accattgctc |
| 541 | aggcatggga aagcatcgct acacatcagc aggaatatca caatacactg tgaaaggcct |
| 601 | ccagttacca actcctgtct ggtttcatgt ccttttctat ctggcagtgg gaataatgtt |
| 661 | tttagtgaac actgttctct gggtgacaat acgtaaagaa ctgaaaagaa agaaaaagtg |
| 721 | gaatttagaa atctctttgg attctggtca tgagaagaag gtaatttcca gccttcaaga |
| 781 | agacagacat ttagaagaag agctgaaatg tcaggaacaa aaagaagaac agctgcagga |
| 841 | aggggtgcac cggaaggagc cccagggggc cacgtaaggc ctcatgggcc cagctttctt |
| 901 | gtac |

A recombinant receptor was previously described, that consists of the extracellular region of human CD64 for high affinity antibody binding, and the transmembrane and intracellular regions of human CD16A for mediating NK cell signal transduction. This receptor, termed CD64/16A, also lacks the membrane proximal ADAM17 cleavage site found in CD16A. See, Snyder K M, et al. Expression of a Recombinant High Affinity IgG Fc Receptor by Engineered NK Cells as a Docking Platform for Therapeutic mAbs to Target Cancer Cells. Front Immunol. 2018 Dec. 6; 9:2873. PMID: 30574146, which is incorporated herein by reference. Suitable high affinity CD64 receptors are described, e.g., in US 2020/0283501, which is incorporated herein by reference.

The CD64 receptor can be encoded by a cDNA that can be transcribed and translated from an expression cassette introduced into a host cell to produce a recombinant cell. The host cell includes a primary leukocyte. In certain embodiments, the immune cell is an NK cell. The expression of CD64 by genetically-engineered leukocytes can increase the recombinant cell's effector function in killing target cells—e.g., tumor cells and virus-infected cells—in combination with therapeutic antibodies compared to the unmodified host cell.

As used herein, a “vector” comprises any genetic element including, without limitation, naked DNA, a phage, transposon, cosmid, episome, plasmid, bacteria, or a virus, which expresses, or causes to be expressed, a desired nucleic acid construct.

In one embodiment, the vector is a non-pathogenic virus. In another embodiment, the vector is a non-replicating virus. In one embodiment, a desirable viral vector may be a retroviral vector, such as a lentiviral vector. In another embodiment, a desirable vector is an adenoviral vector. In still another embodiment, a suitable vector is an adeno-associated viral vector. A variety of adenovirus, lentivirus and AAV strains are available from the American Type Culture Collection, Manassas, Virginia, or available by request from a variety of commercial and institutional sources. Further, the sequences of many such strains are available from a variety of databases including, e.g., PubMed and GenBank.

In one embodiment, a lentiviral vector is used. Among useful vectors are the equine infectious anemia virus and feline as well as bovine immunodeficiency virus, and HIV-based vectors. A variety of useful lentivirus vectors, as well as the methods and manipulations for generating such vectors for use in transducing cells and expressing heterologous genes, e.g., N Manjunath et al, 2009 Adv Drug Deliv Rev., 61(9): 732-745; Porter et al., N Engl J Med. 2011 Aug. 25; 365(8):725-33), among others.

In another embodiment, the vector used herein is an adenovirus vector. Such vectors can be constructed using adenovirus DNA of one or more of any of the known adenovirus serotypes. See, e.g., T. Shenk et al., Adenoviridae: The Viruses and their Replication”, Ch. 67, in FIELD'S VIROLOGY, 6^{th} Ed., edited by B. N Fields et al, (Lippincott Raven Publishers, Philadelphia, 1996), p. 111-2112; 6,083,716, which describes the genome of two chimpanzee adenoviruses; U.S. Pat. No. 7,247,472; WO 2005/1071093, etc. One of skill in the art can readily construct a suitable adenovirus vector to carry and express a nucleotide construct as described herein. In another embodiment, the vector used herein is an adeno-associated virus (AAV) vector. Such vectors can be constructed using AAV DNA of one or more of the known AAV serotypes. See, e.g., U.S. Pat. Nos. 7,803,611; 7,696,179, among others.

In certain embodiments, an expression cassette is delivered via a lipid nanoparticle. The term “lipid nanoparticle” refers to a lipid composition having a typically spherical structure with an average diameter of 10 to 1000 nanometers, e.g. 75 nm to 750 nm, or 100 nm and 350 nm, or between 250 nm to about 500 nm. In some formulations, lipid nanoparticles can comprise at least one cationic lipid, at least one noncationic lipid, and at least one conjugated lipid. Lipid nanoparticles known in the art that are suitable for encapsulating nucleic acids, such as mRNA, may be used. “Average diameter” is the average size of the population of nanoparticles comprising the lipophilic phase and the hydrophilic phase. The mean size of these systems can be measured by standard methods known by the person skilled in the art. Examples of suitable lipid nanoparticles for gene therapy is described, e.g., L. Battaglia and E. Ugazio, J Nanomaterials, Vol 2019, Article ID 283441, pp. 1-22; US2012/0183589A1; and WO 2012/170930 which are incorporated herein by reference in their entirety.

The modified immune cells, e.g., NK cells, further include antibodies attached thereto. Any antibody may be used for which attachment to an NK cell is desirable, for targeting of the NK cell. The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

In one embodiment, an “antibody” refers to an intact immunoglobulin, such as an IgG, or to an antigen binding portion thereof that competes with the intact antibody for specific binding, or a modification thereof, unless otherwise specified. In certain embodiments, an intact antibody is an IgA, IgG, IgM, IgE or IgD. In one embodiment, an intact antibody is an IgG1, IgG2, IgG3 or IgG4. An antibody (e.g., an antibody, an antibody heavy chain, an antibody light chain, or any fragment or modification thereof) comprises three Complementarity-Determining Regions (CDRs, also known as HV, hypervariable regions, namely CDR1, CDR2, CDR3, from N-terminal to C-terminal, or 5′ to 3′ when corresponding nucleic acid sequence is referred to), and four framework regions (FRs, namely FR1, FR2, FR3 and FR4, from N-terminal to C-terminal, or 5′ to 3′ when corresponding nucleic acid sequence is referred to). See, e.g., Janeway, Charles A Jr; Travers, Paul; Walport, Mark; Shlomchik, Mark J (2001). Immunobiology: The Immune System in Health and Disease (5 ed.). New York: Garland Science. ISBN 0-8153-3642-X, which is incorporated herein by its entirety. In the antibody construct, CDRs are arranged non-consecutively, not immediately adjacent to each other, and may be separated by an FR. For example, an antibody may be organized as variable domains/regions (such as FR1CDR1-FR2-CDR2-FR3-CDR3-FR4) following by optional constant regions. As part of the variable chain/region/domain in an antibody construct and T cell receptors generated by B cells and T-cells respectively, CDRs refer to the region where an antigen/epitope specifically binds. The terms “variable region” and “variable domain” are used interchangeably and refer to the portion of an antibody having an amino acid sequence that determines the antigenic specificity of the antibody.

The antibody or fragment includes a monoclonal antibody. Such antibodies can also include a synthetic antibody, a recombinant antibody, a chimeric antibody, a humanized antibody, a human antibody, a CDR-grafted antibody, a multi-specific binding construct that can bind two or more epitopes, a dual specific antibody, a bi-specific antibody, an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one VL (variable region of light chain), one VH (variable region of heavy chain) antigen-binding domain, and one or two constant “effector” domains optionally connected by linker domains, a univalent antibody lacking a hinge region, a single domain antibody, a dual variable domain immunoglobulin (DVD-Ig) binding protein or a nanobody, or any recombinant versions thereof. Definitions and examples of these types of structures are found in the art and in, e.g., U.S. Pat. No. 9,902,772, incorporated by reference herein. In certain embodiments, an antibody also refers to an “antibody mimic” or an “antibody equivalent”.

As used herein, an “antibody mimic” or an “antibody equivalent” refers to affibodies, i.e., a class of engineered affinity proteins, generally small (˜6.5 kDa) single domain proteins that can be isolated for high affinity and specificity to any given target, aptamers, polypeptide molecules that bind to a specific target, an affilin, an affitin, an affimer, an alphabody, an anticalin, an avimer, a DARPin (designed ankyrin repeat proteins), a Fynomer, a Kunitz domain peptide, a monobody, a peptabody and others known in the art.

In one embodiment, an antibody is a minibody which is composed of a single-chain molecule containing one VL, one VH antigen-binding domain, and one or two constant “effector” domains. These elements are connected by linker domains. In still another embodiment, the antibodies useful in the methods and compositions herein are “unibodies”, which are IgG4 molecules from with the hinge region has been removed. The term “recombinant antibody” refers to an antibody that is generated by cloning the immunespecific heavy and light antibody coding sequences into a vector. In one embodiment, the vector is designed for high-yield mammalian expression. The resulting vectors are introduced into expression hosts (e.g., bacteria, virus, yeast or mammalian) for the manufacturing of high-quality functional antibodies. Generally, the coding sequence is not naturally associated with the host cell. Recombinant antibodies have glycosylation patterns that differ from the glycosylation pattern of an antibody having the same sequence if it were to exist in nature. In one embodiment, a recombinant antibody is expressed in a mammalian host cell which is not a human host cell. Notably, individual mammalian host cells have unique glycosylation patterns. Recombinant antibodies can be constructed in vitro by forming an Ig-framework through cloning of scFV or Fab or can be produced from an existing hybridoma. In hybridoma-based recombinant antibody generation, mouse, rat, and rabbit models are commonly used. However, as long as the appropriate oligonucleotide primers are available, recombinant antibodies can be developed from any species.

In certain embodiments, the antibodies are anti-HIV antibodies. In certain embodiments, the antibodies are broadly neutralizing anti-HIV-1 antibodies. A broadly neutralizing antibody (bnAb) is an antibody that neutralizes many different genetic variants of HIV. Multiple bnAbs that exhibit breath and potency against epitopes on the HIV-1 envelope trimer are currently being assessed in clinical trials for HIV-1 prevention, treatment as well as remission induction. These antibodies are useful herein. The targeted areas on HIV-1 envelope include the CD4-binding site (CD4bs) on gp120 [antibodies VRC01; VRC01-LS, 3BNC117, 3BNC117-LS, VRC07-523LS and N6LS]; the glycan-dependent epitopes on V1/V2 (antibodies PGDM1400 and CAP256V2LS as well as V3 loops (antibodies 10-1074, 10-1074-LS, PGT121 and PGT121.414.LS); the linear epitopes in the membrane-proximal external region (MPER) on gp41 [antibody 10E8VLS]. Other bnAbs with high potency and breadth that have not yet entered clinical trials include antibodies targeting the gp120-gp41 interface and the N49 lineage of CD4bs bnAbs. See, e.g., Wu X, Yang Z Y, Li Y, Hogerkorp C M, Schief W R, Seaman M S, et al. Rational Design of Envelope Identifies Broadly Neutralizing Human Monoclonal Antibodies to HIV-1. Science (2010) 329(5993):856-61. doi: 10.1126/science.1187659; Ko S Y, Pegu A, Rudicell R S, Yang Z Y, Joyce M G, Chen X, et al. Enhanced Neonatal Fc Receptor Function Improves Protection Against Primate SHIV Infection. Nature (2014) 514(7524):642-5. doi: 10.1038/nature13612; Scheid J F, Mouquet H, Ueberheide B, Diskin R, Klein F, Oliveira T Y, et al. Sequence and Structural Convergence of Broad and Potent HIV Antibodies That Mimic CD4 Binding. Science (2011) 333(6049):1633-7. doi: 10.1126/science.1207227; Gautam R, Nishimura Y, Gaughan N, Gazumyan A, Schoofs T, Buckler-White A, et al. A Single Injection of Crystallizable Fragment Domain-Modified Antibodies Elicits Durable Protection From SHIV Infection. Nat Med (2018) 24(5):610-6. doi: 10.1038/s41591-018-0001-2; Rudicell R S, Kwon Y D, Ko S Y, Pegu A, Louder M K, Georgiev I S, et al. Enhanced Potency of a Broadly Neutralizing HIV-1 Antibody In Vitro Improves Protection Against Lentiviral Infection In Vivo. J Virol (2014) 88(21):12669-82. doi: 10.1128/JVI.02213-14; Huang J, Kang B H, Ishida E, Zhou T, Griesman T, Sheng Z, et al. Identification of a CD4-Binding-Site Antibody to HIV That Evolved Near-Pan Neutralization Breadth. Immunity (2016) 45(5):1108-21. doi: 10.1016/j.immuni.2016.10.027; Sok D, van Gils M J, Pauthner M, Julien J P, Saye-Francisco K L, Hsueh J, et al. Recombinant HIV Envelope Trimer Selects for Quaternary-Dependent Antibodies Targeting the Trimer Apex. Proc Natl Acad Sci USA (2014) 111(49):17624-9. doi: 10.1073/pnas.1415789111; Doria-Rose N A, Schramm C A, Gorman J, Moore P L, Bhiman J N, DeKosky B J, et al. Developmental Pathway for Potent V1V2-Directed HIV-Neutralizing Antibodies. Nature (2014) 509(7498):55-62. doi: 10.1038/nature13036; Mouquet H, Scharf L, Euler Z, Liu Y, Eden C, Scheid J F, et al. Complex-Type N-Glycan Recognition by Potent Broadly Neutralizing HIV Antibodies. Proc Natl Acad Sci USA (2012) 109(47):E3268-77. doi: 10.1073/pnas.1217207109; Walker L M, Huber M, Doores K J, Falkowska E, Pejchal R, Julien J P, et al. Broad Neutralization Coverage of HIV by Multiple Highly Potent Antibodies. Nature (2011) 477(7365):466-70. doi: 10.1038/nature10373; Huang J, Ofek G, Laub L, Louder M K, Doria-Rose N A, Longo N S, et al. Broad and Potent Neutralization of HIV-1 by a gp41-Specific Human Antibody. Nature (2012) 491(7424):406-12. doi: 10.1038/nature11544; Kwon Y D, Georgiev I S, Ofek G, Zhang B, Asokan M, Bailer R T, et al. Optimization of the Solubility of HIV-1-Neutralizing Antibody 10E8 Through Somatic Variation and Structure-Based Design. J Virol (2016) 90(13):5899-914. doi: 10.1128/JVI.03246-15; McCoy L E, Burton D R. Identification and Specificity of Broadly Neutralizing Antibodies Against HIV. Immunol Rev (2017) 275(1):11-20. doi: 10.1111/imr.12484; Sajadi M M, Dashti A, Rikhtegaran Tehrani Z, Tolbert W D, Seaman M S, Ouyang X, et al. Identification of Near-Pan-Neutralizing Antibodies Against HIV-1 by Deconvolution of Plasma Humoral Responses. Cell (2018) 173(7):1783-1795 e14. doi: 10.1016/j.cell.2018.03.061; Liu Q, Zhang P, Miao H, Lin Y, Kwon Y D, Kwong P D, et al. Rational Engraftment of Quaternary-Interactive Acidic Loops for Anti-HIV-1 Antibody Improvement. J Virol (2021) 95(12):e00159-21. doi: 10.1128/JVI.00159-21, all of which are incorporated herein by reference.

In certain embodiments, the anti-HIV antibodies are 10-1074 antibodies, or variants thereof. See, WO 2020/056145 and Scharf et al, Complex-type N-glycan recognition by potent broadly neutralizing HIV antibodies. Proc Natl Acad Sci USA. 2012 Nov. 20; 109(47):E3268-77. doi: 10.1073/pnas.1217207109. Epub 2012 Oct. 30. PMID: 23115339, and Tables 2 and 3. In other embodiments, the anti-HIV antibodies are selected from 10-259, 10-303, 10-410, 10-847, 10-996, 10-1121, 10-1130, 10-1146, 10-1341, 10-1369, 10-1074GM, GL, 10E8, 12A12, 12A21, 2F5, 2G12, 35022, 3BC176, 3BNC117, 3BNC55, 3BNC60, 3BNC62, 447-52D, 4E10, 5H/I1-BMV-D5, 8ANC195, b12, CAP256-VRC26.01, CAP256-VRC26.02, CAP256-VRC26.03, CAP256-VRC26.04, CAP256-VRC26.05, CAP256-VRC26.06, CAP256-VRC26.07, CAP256-VRC26.08, CAP256-VRC26.09, CAP256-VRC26.10, CAP256-VRC26.il, CAP256-VRC26.12, CHOI, CH02, CH03, CHO4, CH103, HGN194, HJ16, HK20, M66.6, NIH45-46, PCDN-33A, PCDN-33B, PCDN-38A, PG9, PG16, PGDM1400, PGDM1401, PGDM1402, PGDM1403, PGDM1404, PGDM1405, PGDM1406, PGDM1407, PGDM1408, PGDM1409, PGDM1410, PGDM1411, PGDM1412, PGT121, PGT122, PGT123, PGT125, PGT126, PGT127, PGT128, PGT130, PGT131, PGT135, PGT136, PGT137, PGT141, PGT142, PGT143, PGT145, PGT151, PGT152, VRC-CH30, VRC-CH31, VRC-CH32, VRC-CH33, VRC-CH34, VRC-PG04, VRC-CH04b, VRC-PG20, VRC01, VRC02, VRC03, VRC07, VRC23, or Z13.

In certain embodiments, the anti-HIV antibody is 3BNC117. 3BNC117 is a next-generation bNAb that targets the CD4 binding site on HIV envelope gpl60. It is a recombinant human IgG1 kappa monoclonal antibody cloned from an HIV-infected viremic controller. A long-acting version of 3BNC117 is known as 3BNC117-LS. 3BNC117 is described in U.S. Pat. No. 9,783,594, which is included by reference.

In certain embodiments, the antibodies are SARS-COV-2 antibodies. SARS-CoV2 is the causative agent of COVID-19 and antibodies specific for this virus have been described. Examples of IgG antibodies which have been described as being useful for binding the spike protein of human ACE2 of SARS-CoV2 and having neutralizing activity include, e.g., LY-CoV555 (Eli Lilly), TY027 (Tychon), STI-1499 and STI-2020 (COVI-GUARD; Sorrento), 80R, ADI055689/56046 (Adimab) (Renn et al., Trends in Pharmacological Sciences, 2020); BD-217, BD-218, BD-236 (Cao et al., Cell, 182, 73-84 (2020)). Examples of IgG antibodies which have been described as being useful for binding the receptor binding domain (RBD) of human ACE2 of SARS-COV2 and having neutralizing activity include, e.g., COV2-2196, COV2-2130, COV2-2165 (Zost et al., Nature, 584, 443-465 (2020)); BD-361, BD-368, BD-368-2 (Cao et al., Cell, 182, 73-84 (2020)); B38, H4 (Y. Wu et al., Science 10.1126/science.abc2241 (2020); Jahanshahlu and Rezaei, Biomedicine and Pharmacotherapy 129 (2020)); S309, S315, S304 (Pinto et al., Nature, 583, 290-311 (2020)); CC6.29, CC6.30, CC6.33, CC12.1, CC12.3(Rogers et al., Science 369, 956-963 (2020)); JS016(Eli Lilly), CA1, CB6-LALA, P2C-1F11/P2B-2F6/P2A-1A3, 311mab-31B5311/32D4, COVA 2-15, 414-1, (Renn et al., Trends in Pharmacological Sciences, 2020). Examples of IgG antibodies which have been described as being useful for binding the spike protein of human ACE2 of SARS-COV1 and having neutralizing activity include, e.g., m396 and CR3104 (Prabakaran et al., Journal of Biological Chemistry, 281, 15829-15836 (2006); ter Meulen et al., PLoS, 3, 7 (2006)). Examples of IgG antibodies which have been described as being useful for binding either RBD or spike protein of human ACE2 of both SARS-COV1 and SARS-CoV2 and having neutralizing activity include, e.g., CR3022 and 47D11 (Wang et al., Nature Communications, 11, www.nature.com/naturecommunications (2020)).

Exemplary anti-CoV-S antibodies include H4sH15188P, H1H15188P, HTH15211P, H1H15177P, H4sH15211P, H1H15260P2, H1H15259P2, H1H15260P2, H1H152P2, H62PH25, H44sH15, H4sH15188P, H1H15188P, HTH15211P, H1H15177P, and H44sH152, as described in International Patent Application Publication No. WO/2015/179535. H1H15237P2, H1H15208P, H1H15228P2, H1H15233P2, H1H15264P2, H1H15231P2, H1H15253P2, H1H15215P, and H1H15249P2, or antigen binding fragments thereof, e.g. A light chain immunoglobulin (eg, VL or its light chain) comprising any of CDR-L1, CDR-L2, and CDR-L3, and a heavy immunoglobulin comprising CDR-H1, CDR-H2, and CDR-H3 of chain (eg, VH or its heavy chain).

In certain embodiments, the antibodies are cancer antibodies. Non-limiting examples of anti-cancer molecules include immune checkpoint inhibitors (e.g., CTLA-4 antibodies, PD-1 antibodies, PDL-1 antibodies), cytotoxic agents (e.g., Cly A, FASL, TRAIL, TNF-alpha), immunostimulatory cytokines and co-stimulatory molecules (e.g., OX40, CD28, ICOS, CCL21, IL-2, IL-18, IL-15, IL-12, IFN-gamma, IL-21, TNFs, GM-CSF), antigens and antibodies (e.g., tumor antigens, neoantigens, CtxB-PSA fusion protein, CPV-OmpA fusion protein, NY-ESO-1 tumor antigen, RAF1, antibodies against immune suppressor molecules, anti-VEGF, Anti-CXR4/CXCL12, anti-GLP1, anti-GLP2, anti-galectinl, anti-galectin3, anti-Tie2, anti-CD47, antibodies against immune checkpoints, antibodies against immunosuppressive cytokines and chemokines), DNA transfer vectors (e.g., endostatin, thrombospondin-1, TRAIL, SMAC, Stat3, Bcl2, FLT3L, GM-CSF, IL-12, AFP, VEGFR2), and enzymes (e.g., E. coli CD, HSV-TK).

In one embodiment, an antibody includes neutralizing antibodies against a viral pathogen. Such anti-viral antibodies may include anti-influenza antibodies directed against one or more of Influenza A, Influenza B, and Influenza C. The 20 type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7. Other target pathogenic viruses include, without limitation, arenaviruses (including funin, 25 machupo, and Lassa), filoviruses (including Marburg and Ebola), hantaviruses, picornoviridae (including rhinoviruses, echovirus), coronaviruses, paramyxovirus, morbillivirus, respiratory synctial virus, togavirus, coxsackievirus, JC virus, parvovirus B19, parainfluenza, adenoviruses, reoviruses, variola (Variola major (Smallpox)) and Vaccinia (Cowpox) from the poxvirus family, and varicella-zoster (pseudorabies). Viral hemorrhagic 30 fevers are caused by members of the arenavirus family (Lassa fever) (which family is also associated with Lymphocytic choriomeningitis (LCM)), filovirus (ebola virus), and hantavirus (puremala). The members of picornavirus (a subfamily of rhinoviruses), are associated with the common cold in humans. The coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The human respiratory coronaviruses have been putatively associated 5 with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which 10 includes respiratory syncytial virus (RSV). The parvovirus family includes feline parvovirus (feline enteritis), feline panleucopeniavirus, canine parvovirus, and porcine parvovirus. The adenovirus family includes viruses (EX, AD7, ARD, O.B.) which cause respiratory disease. Thus, in certain embodiments, an antibody may include an anti-ebola antibody, e.g., 2G4, 4G7, 13C6, an anti-influenza antibody, e.g., FI6, 15 CR8033, and anti-RSV antibody, e.g, palivizumab, motavizumab.

A neutralizing antibody construct against a bacterial pathogen may also be selected for use in the present invention. In one embodiment, the neutralizing antibody construct is directed against the bacteria itself. In another embodiment, the neutralizing antibody construct is directed against a toxin produced by the bacteria. Examples of airborne bacterial pathogens include, e.g., 20 Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), 25 Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria 30 (diphtheria), Mycoplasma pneumoniae (pneumonia).

In one embodiment, antibody includes antibodies, and particularly neutralizing antibodies against a bacterial pathogen such as the causative agent of anthrax, a toxin produced by Bacillius anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther 5 Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used as described herein. Antibodies against infectious diseases may be caused by parasites or by fungi, including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

In another embodiment, an antibody includes antibodies, and particularly neutralizing antibodies, against pathogenic factors of diseases such as Alzheimer's disease (AD), Parkinson's disease (PD), Rheumatoid arthritis (RA), Irritable bowel syndrome (IBS), chronic obstructive pulmonary disease (COPD), cancers, tumors, systemic sclerosis, asthma and other diseases. Such antibodies may be, without limitation, e.g., alpha-synuclein, anti-20—vascular endothelial growth factor (VEGF) (anti-VEGF), anti-VEGFA, anti-PD-1, antiPDLI, anti-CTLA-4, anti-TNF-alpha, anti-IL-17, anti-IL-23, anti-IL-21, anti-IL-6, anti-IL-6 receptor, anti-IL-5, anti-IL-7, anti-Factor XII, anti-IL-2, anti-HIV, anti-IgE, anti-tumour necrosis factor receptor-1 (TNFR1), anti-notch 2/3, anti-notch 1, anti-OX40, anti-erb-b2 receptor tyrosine kinase 3 (ErbB3), anti-ErbB2, anti-beta cell maturation antigen, anti-B 25 lymphocyte stimulator, anti-CD20, anti-HER2, anti-granulocyte macrophage colony-stimulating factor, anti-oncostatin M (OSM), anti-lymphocyte activation gene 3 (LAG3) protein, anti-CCL20, anti-serum amyloid P component (SAP), anti-prolyl hydroxylase inhibitor, anti-CD38, anti-glycoprotein IIb/IIIa, anti-CD52, anti-CD30, anti-IL-ibeta, antiepidermal growth factor receptor, anti-CD25, anti-RANK ligand, anti-complement system 30 protein C5, anti-CDIIa, anti-CD3 receptor, anti-alpha-4 (a4) integrin, anti-RSV F protein, and anti-integrin a407. Still other pathogens and diseases will be apparent to one of skill in the art. Other suitable antibodies may include those useful for treating Alzheimer's Disease, such as, e.g., anti-beta-amyloid (e.g., crenezumab, solanezumab, aducanumab), anti-betaamyloid fibril, anti-beta-amyloid plaques, anti-tau, a bapineuzamab, among others.

### Compositions & Methods

In one aspect, a modified human primary NK cell is provided that comprises a nucleic acid sequence that encodes a high affinity CD64 Fc receptor, wherein the cell further has antibodies attached. In certain embodiments, the antibodies are anti-HIV bnAbs.

A modified immune cell, e.g., NK cell, is one that has been transduced or transfected with one of the above-described vectors carrying a nucleic acid construct encoding the high affinity CD64 receptor. Desirably, the cell is a primary NK cell. In certain embodiments, the cell is obtained from the same mammalian subject into whom the modified primary cell is administered or from another member of the mammalian species. In one embodiment, the cell is an autologous natural killer (NK) cell obtained from the subject or from a bone marrow transplant match for the subject. The cell is generally obtained by apheresis, and transfected or transduced with the selected nucleic acid construct to express the protein in vivo.

In certain embodiments, the modified immune cell is cultured prior to administration to the subject. In some embodiments, the culture medium is supplemented with one or more cytokines. Various human cytokines are known in the art and include those of Table 1, below.

| Name | Synonym(s) |
|---|---|
| Interleukins | |
| IL-1-like | |
| IL-1α | hematopoietin-1 |
| IL-1β | catabolin |
| IL-1RA | IL-1 receptor |
| | antagonist |
| IL-18 | interferon-γ |
| | inducing |
| | factor |
| Common g | |
| chain (CD132) | |
| IL-2 | T cell growth |
| | factor |
| IL-4 | BSF-1 |
| IL-7 | |
| IL-9 | T cell growth |
| | factor P40 |
| IL-13 | P600 |
| IL-15 | |
| Common b | |
| chain (CD131) | |
| IL-3 | multipotential |
| | CSF, MCGF |
| IL-5 | BCDF-1 |
| Also related | |
| GM-CSF | CSF-2 |
| IL-6-like | |
| IL-6 | IFN-β2, BSF-2 |
| IL-11 | AGIF |
| Also related | |
| G-CSF | CSF-3 |
| IL-12 | NK cell |
| | stimulatory |
| | factor |
| LIF | leukemia |
| | inhibitory |
| | factor |
| OSM | oncostatin M |
| IL-10-like | |
| IL-10 | CSIF |
| IL-20 | |
| Others | |
| IL-14 | HMW-BCGF |
| IL-16 | LCF |
| IL-17 | CTLA-8 |
| Interferons | |
| IFN-α | |
| IFN-β | |
| IFN-γ | |
| TNF | |
| CD154 | CD40L, TRAP |
| LT-β | |
| TNF-α | cachectin |
| TNF-β | LT-α |
| 4-1BBL | |
| APRIL | TALL-2 |
| CD70 | CD27L |
| CD153 | CD30L |
| CD178 | FasL |
| GITRL | |
| LIGHT | |
| OX40L | |
| TALL-1 | |
| TRAIL | Apo2L |
| TWEAK | Apo3L |
| TRANCE | OPGL |
| TGF-β | |
| TGF-β1 | TGF-β |
| TGF-β2 | |
| TGF-β3 | |
| Miscellaneous | |
| hematopoietins | |
| Epo | erythropoietin |
| Tpo | MGDF |
| Flt-3L | |
| SCF | stem cell factor, |
| | c-kit ligand |
| M-CSF | CSF-1 |
| MSP | Macrophage |
| | stimulating |
| | factor, MST-1 |

Concentrations of cytokines may be established based on culture conditions, but may range from about 5 ng/mL to about 500 ng/mL, including endpoints and all numbers therebetween. Cytokine concentrations may include 5 ng/mL, 10 ng/mL, 15 ng/mL, 20 ng/mL, 25 ng/mL, 30 ng/mL, 35 ng/mL, 40 ng/mL, 45 ng/mL, 50 ng/mL, 55 ng/mL, 60 ng/mL, 65 ng/mL, 70 ng/mL, 75 ng/mL, 80 ng/mL, 85 ng/mL, 90 ng/mL, 95 ng/mL, 100 ng/mL, 105 ng/mL, 110 ng/mL, 115 ng/mL, 120 ng/mL, 125 ng/mL, 130 ng/mL, 135 ng/mL, 140 ng/mL, 145 ng/mL, 150 ng/mL, 155 ng/mL, 160 ng/mL, 165 ng/mL, 170 ng/mL, 175 ng/mL, 180 ng/mL, 185 ng/mL, 190 ng/mL, 195 ng/mL, 200 ng/mL, 205 ng/mL, 210 ng/mL, 215 ng/mL, 220 ng/mL, 225 ng/mL, 230 ng/mL, 235 ng/mL, 240 ng/mL, 245 ng/mL, 250 ng/mL, 255 ng/mL, 260 ng/mL, 265 ng/mL, 270 ng/mL, 275 ng/mL, 280 ng/mL, 285 ng/mL, 290 ng/mL, 295 ng/mL, 300 ng/mL, 305 ng/mL, 310 ng/mL, 315 ng/mL, 320 ng/mL, 325 ng/mL, 330 ng/mL, 335 ng/mL, 340 ng/mL, 345 ng/mL, 350 ng/mL, 355 ng/mL, 360 ng/mL, 365 ng/mL, 370 ng/mL, 375 ng/mL, 380 ng/mL, 385 ng/mL, 390 ng/mL, 395 ng/mL, 400 ng/mL, 405 ng/mL, 410 ng/mL, 415 ng/mL, 420 ng/mL, 425 ng/mL, 430 ng/mL, 435 ng/mL, 440 ng/mL, 445 ng/mL, 450 ng/mL, 455 ng/mL, 460 ng/mL, 465 ng/mL, 470 ng/mL, 475 ng/mL, 480 ng/mL, 485 ng/mL, 490 ng/mL, 495 ng/mL, and 500 ng/mL. The relevant concentration for each cytokine used may be determined individually.

The cells are cultured for the desired amount of time. In one embodiment, the cells are cultured for 1 day to 21 days. In another embodiment, the cells are cultured for 2, 3, 4, 5, 6, 7, 8, 9, 10, 11, 12, 13, 14, 15, 16, 17, 18, 19, 20, or 21 days, or any time therebetween.

The pharmaceutical compositions and modified NK cells described herein are useful in cell therapies, both autologous and allogeneic. Autologous cell therapy (ACT) is a therapeutic intervention that uses an individual's cells, which are cultured and expanded outside the body, and reintroduced into the donor. Advantages of such an approach include the minimization of risks from systemic immunological reactions, bio-incompatibility, and disease transmission associated with grafts or cells not cultivated from the individual. Thus, in one embodiment, the methods include removal of the source blood cells from the donor.

In one aspect, a method of treating HIV-1 infection in a subject is provided. In one embodiment, the method includes administering a therapeutically effective amount of a pharmaceutical composition comprising modified NK cells as described herein. In one embodiment, the therapeutically effective amount is about 1×10⁵ to about 1×10¹⁴ cells, preferably 1×10⁸ to 1×10¹¹ cells, including endpoints and all integers therebetween. In another embodiment, the effective amount is about 5×10⁸ to 2×10¹⁰ cells, including endpoints and all integers therebetween.

In another aspect, a method of inducing or augmenting antibody-dependent cellular cytotoxicity (ADCC) in a subject is provided. Antibody-dependent cellular cytotoxicity (ADCC), also called antibody-dependent cell-mediated cytotoxicity, is an immune mechanism through which Fc receptor-bearing effector cells can recognize and kill antibody-coated target cells expressing tumor- or pathogen-derived antigens on their surface. In one embodiment, the method includes administering a therapeutically effective amount of a pharmaceutical composition comprising modified NK cells as described herein. In one embodiment, the therapeutically effective amount is about 1×10⁵ to about 1×10¹⁴ cells, preferably 1×10⁸ to 1×10¹¹ cells, including endpoints and all integers therebetween. In another embodiment, the effective amount is about 5×10⁸ to 2×10¹⁰ cells, including endpoints and all integers therebetween.

In one aspect, a therapeutically effective amount of modified immune cells as described herein, is provided for use in treating HIV-1 infection in a subject.

The treatments may include various “unit doses.” Unit dose is defined as containing a predetermined quantity of the therapeutic composition (modified cell composition) calculated to produce the desired responses in association with its administration, i.e., the appropriate route and treatment regimen. The quantity to be administered, and the particular route and formulation, are within the skill of those in the clinical arts. Also of import is the subject to be treated, in particular, the state of the subject and the protection desired. A unit dose need not be administered as a single injection but may comprise continuous infusion over a set period of time.

In certain embodiments, the composition is given in a single dose or multiple doses. The single dose may be administered daily, or multiple times a day, or multiple times a week, or monthly or multiple times a month. In a further embodiment, the composition is given in a series of doses. The series of doses may be administered daily, or multiple times a day, weekly, or multiple times a week, or monthly, or multiple times a month.

Dosages and administration regimen can be adjusted depending on the age, sex, physical condition of administered as well as the benefit of the conjugate and side effects in the patient or mammalian subject to be treated and the judgment of the physician, as is appreciated by those skilled in the art.

In certain embodiments, it is desirable to combine therapy with the modified primary immune cell described herein, with another treatment for HIV-1 infection, including anti-retroviral therapy. The term “anti-retroviral therapy” or “ART” refers to treatment of individuals infected with human immunodeficiency virus (HIV) using anti-HIV drugs. The standard treatment consists of a combination of at least three drugs (often called “highly active antiretroviral therapy” or HAART) that suppress HIV replication. Antiretroviral medicines that are often used to treat HIV include: Nucleoside/nucleotide reverse transcriptase inhibitors, also called nucleoside analogs, such as abacavir, emtricitabine, and tenofovir. These medicines are often combined for best results. Nonnucleoside reverse transcriptase inhibitors (NNRTIs), such as efavirenz, etravirine, and nevirapine are used. Protease inhibitors (PIs), such as atazanavir, darunavir, and ritonavir are used. Entry inhibitors, such as enfuvirtide and maraviroc are used. Integrase inhibitors, such as dolutegravir and raltegravir are used. In one embodiment, ART is a combination of drugs efavirenz, tenofovir, and emtricitabine. Other combinations, without limitation, include: Dolutegravir, abacavir and lamivudine; Dolutegravir, tenofovir and emtricitabine; elvitegravir, cobicistat and tenofovir; and emtricitabine, raltegravir, tenofovir and emtricitabine; or ritonavir-boosted darunavir, tenofovir and emtricitabine.

As used herein, the term “subject” or “patient” refers to a male or female mammal, preferably a human. However, the mammalian subject can also be a veterinary or farm animal, a domestic animal or pet, and animals normally used for clinical research. In one embodiment, the subject of these methods and compositions is a human.

The term “cancer” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. The compositions and methods described herein are useful for treatment of cancer and tumor cells, i.e., both malignant and benign tumors. Thus, in various embodiments of the methods and compositions described herein, the cancer can include, without limitation, breast cancer, lung cancer, prostate cancer, colorectal cancer, esophageal cancer, stomach cancer, bladder cancer, pancreatic cancer, kidney cancer, cervical cancer, liver cancer, ovarian cancer, and testicular cancer.

As used herein the term “pharmaceutically acceptable carrier” or “diluent” is intended to include any and all solvents, dispersion media, coatings, antibacterial and antifungal agents, isotonic and absorption delaying agents, adjuvants and the like, compatible with administration to humans. In one embodiment, the diluent is saline or buffered saline. The term “a” or “an”, refers to one or more, for example, “an anti-tumor T cell” is understood to represent one or more anti-tumor T cells. As such, the terms “a” (or “an”), “one or more,” and “at least one” is used interchangeably herein. The term “about” is used herein to modify a reference value and to include all values 0.010% of that value up to values of +10% of the reference value, and all numbers within and including these endpoints, e.g., +0.5%, +1%, +5%, etc. Various embodiments in the specification are presented using “comprising” language, which is inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention.

### EXAMPLES

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitutions of reagents that are chemically or physiologically related for the reagents described herein are anticipated to produce the same or similar results. All such similar substitutes and modifications are apparent to those skilled in the art and fall within the scope of the invention.

### Example 1: CD64 Gene Modified Primary Human NK Cells Loaded with Anti-Hiv Bnabs Trigger High Adcc

We transduced purified NK cells from control donors with a lentivirus expressing human CD64 and expanded them ex vivo for multiple weeks in the presence of various cytokines. CD64 positive or Control NK cells were then pre-loaded with a fluorescently conjugated HIV-specific BNAb, washed, and then followed over time to measure their ability to bind and retain antibody. To measure their ADCC CD107a degranulation function, CD64 transduced or control NK cells were FACS sorted to high purity and incubated with autologous HIV-1 infected CD4⁺ primary T cells in the presence or absence of HIV-specific BNAbs which were added either to the NK effectors (to measure Ab retention) or Target cells (as a positive control).

### Results

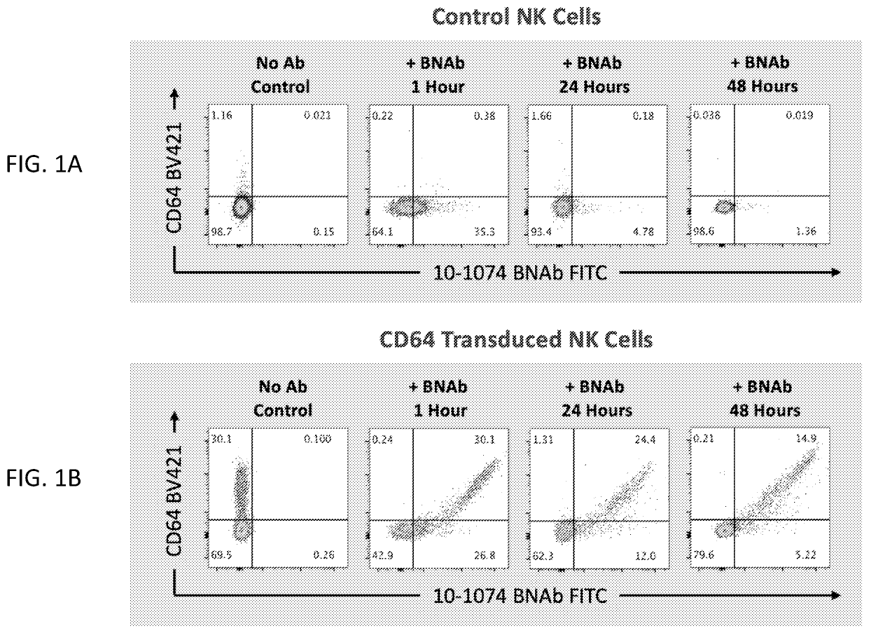

Following pre-loading with HIV-specific BNAbs, CD64 transduced NK cells could successfully retain BNAbs for several days as compared to control NK cells (FIG. 1A and FIG. 1B).

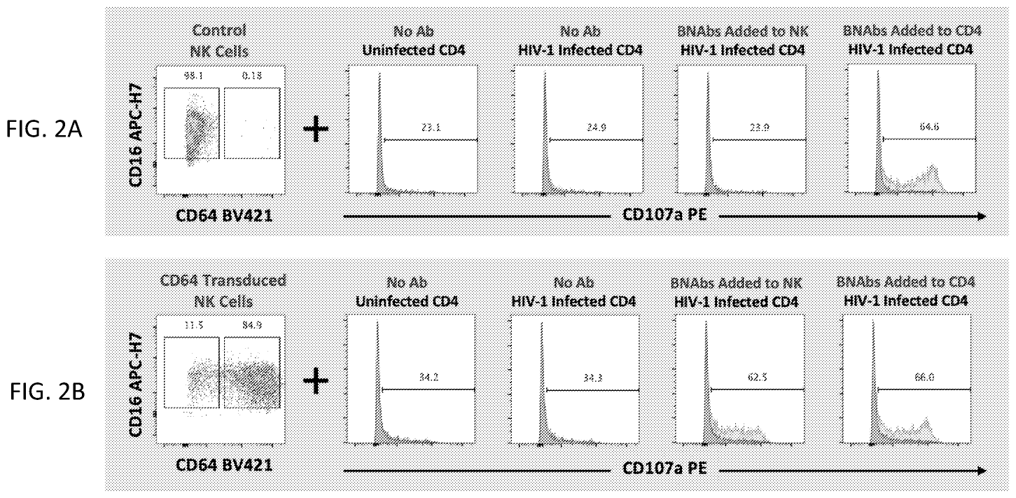

After FACS sorting, CD64 transduced NK cells showed a significant increase in ADCC-triggered CD107a degranulation capacity against autologous HIV-1 infected CD4+ primary T cells compared to control NK cells after pre-loading with HIV-specific BNAbs (FIG. 2A and FIG. 2B).

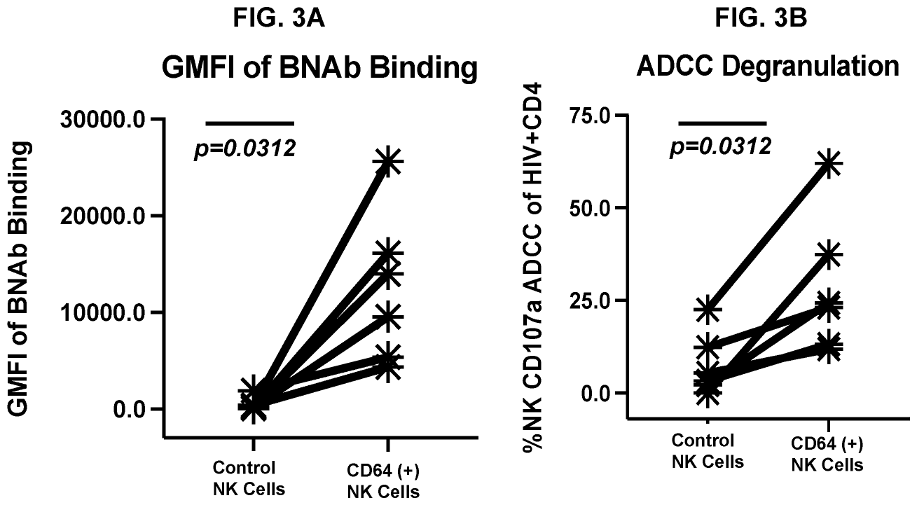

Composite data from experiments where BNAb binding by CD64 transduced and control NK cells was determined at 1 hour post incubation with BNAbs and after washing to remove unbound BNAbs (FIG. 3A) as well as composite data from experiments measuring ADCC CD107a degranulation by CD64 transduced and control NK cells against HIV-1 infected CD4+ primary T cells after HIV-specific BNAb pre-loading demonstrates a statistically significant ability of CD64 transduced NK cells to bind the BNAbs of interest (FIG. 3A) and use them to trigger NK ADCC degranulation (FIG. 3B).

### Example 2: Bnab Pre-Loaded Transduced Autologous CD64 Positive NK Cells, but not Bnab Pre-Loaded Transduced Autologous CD64 Positive CD8 T Cells, are Able to Eliminate HIV-1 Infected CD4 T Cells

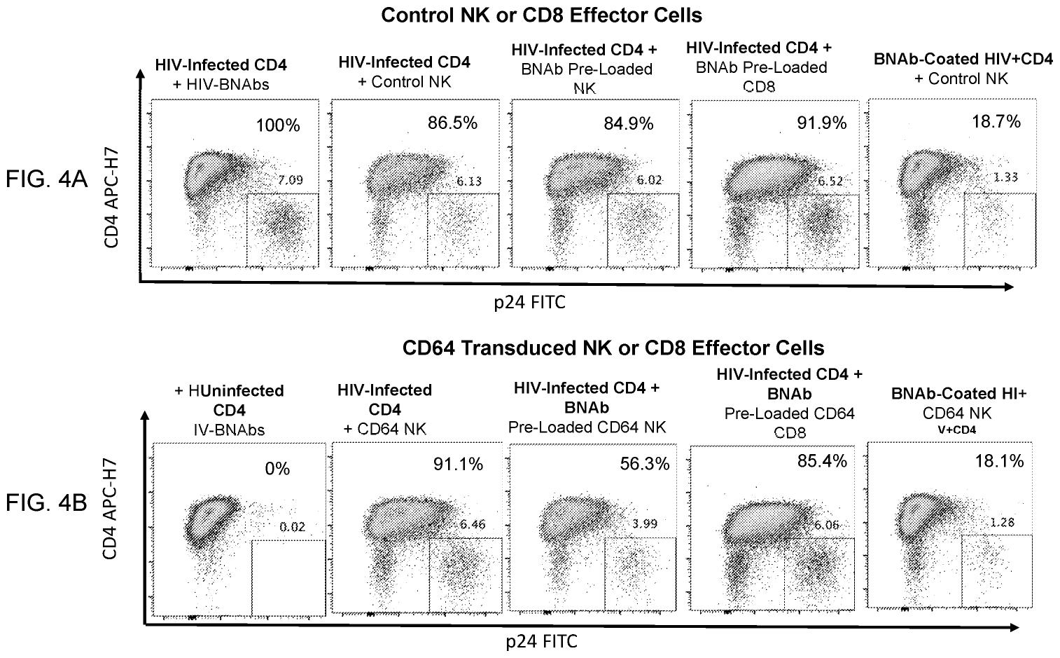

An HIV-1 Elimination Assay was performed where HIV-1 NL4-3 infected CD4⁺ primary T cells were incubated in the presence of 100 mg/mL of HIV-specific BNAbs or incubated with control autologous NK cells in the absence of antibody for 18 hours. Control autologous NK cells or control autologous CD8 T cells were pre-loaded with HIV-specific BNAbs, washed and then incubated with HIV-1 NL4-3 infected CD4⁺ primary T cells for 18 hours. Additionally, HIV-specific BNAbs were added to the HIV-1 NL4-3 infected CD4⁺ primary T cells during the entire 18-hour incubation in the presence of control NK cells (positive control) (FIG. 4A).

A separate HIV-1 Elimination Assay was performed where uninfected CD4+ primary T cells were incubated in the presence of HIV-specific BNAbs or incubated with CD64 transduced and sorted autologous NK cells in the absence of antibody for 18 hours. CD64 transduced and sorted autologous NK cells or CD64 transduced and sorted autologous CD8 T cells were pre-loaded HIV-specific BNAbs, washed and then incubated with HIV-1 NL4-3 infected CD4⁺ primary T cells for 18 hours. As a positive control, HIV-specific were added to the HIV-1 NL4-3 infected CD4⁺ primary T cells during the entire 18-hour incubation in the presence of CD64 transduced and sorted autologous NK cells (FIG. 4B).

### Results

These elimination assays showed that BNAb pre-loaded CD64 transduced NK cells were able to eliminate HIV-1 infected autologous CD4 primary T cells. However, BNAb pre-loaded CD 8 cells were not able to eliminate HIV-1 infected autologous CD4 primary T cells.

### Example 3: Control NK Cells and CD64 Transduced NK Cells Express the Trail Ligand while HIV-1 Infected CD4 T Cells Also Express the Trail Death Receptors DR4 and DR5

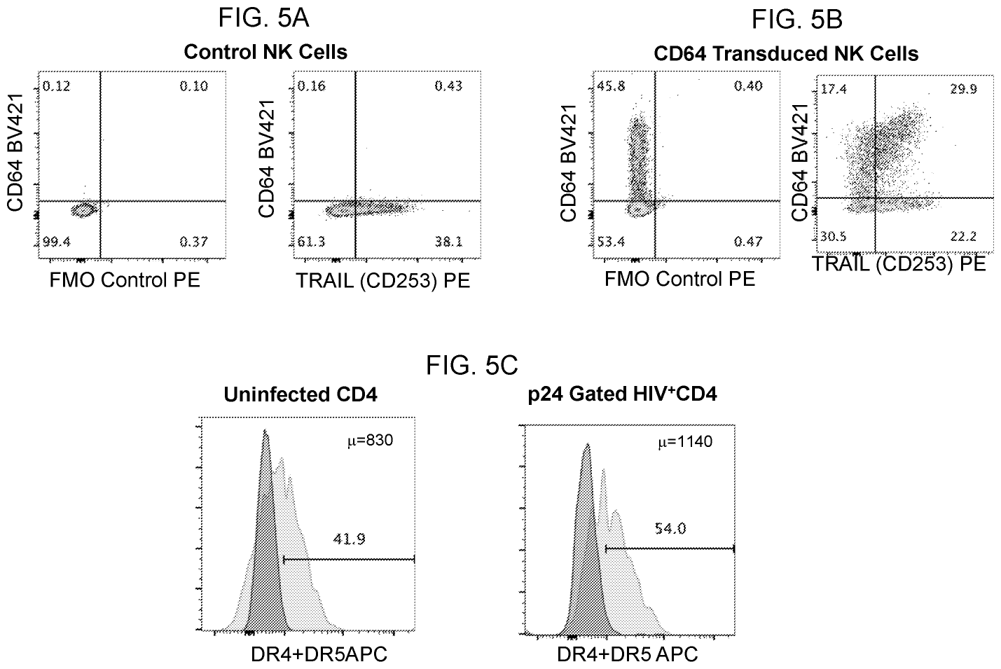

Flow cytometry was used on cytokine stimulated control NK cells (FIG. 5A) and CD64 transduced NK cells (FIG. 5B) to monitor the expression of the Trail Death Receptor Ligand. Flow cytometry was also used on autologous uninfected and HIV-1 infected CD4 primary T cells to monitor expression of the Trail Death Receptors DR4 and DR5 (FIG. 5C).

### Results

The flow cytometry data showed the control NK cells and CD64 transduced NK cells express the Trail Death Receptor Ligand. Further, HIV-1 infected CD4 T cells were shown to express the Trail Death Receptors DR4 and DR5.

### Example 4: CD64 Transduced NK Cells are Able to Proliferate Over Time which Allows for Durable Expression of the CD64 High-Affinity Fc Receptor

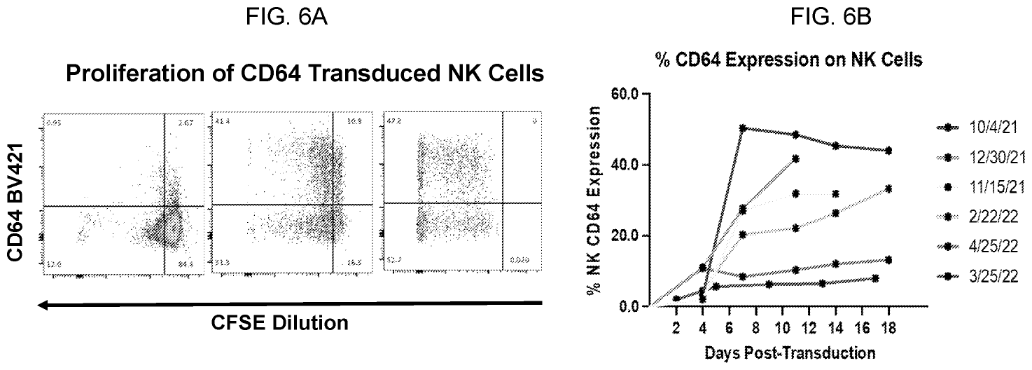

Human NK cells were purified and stimulated with cytokines to trigger NK activation. Cytokine stimulated NK cells were labeled to measure NK cell proliferation then transduced with a Lentivirus encoding the high-affinity Fc receptor CD64. CFSE dilution and CD64 expression was monitored on transduced NK cells by flow cytometry over time (FIG. 6A).

### Results

CD64 transduced and non-transduced NK cells exhibited equivalent proliferative capacity over time (FIG. 6A). Composite data shows CD64 expression peaked on NK cells around Day 7 and was stably maintained on NK cells over time (FIG. 6B). The ability of both control NK cells and CD64 transduced NK cells to proliferate equally allows for durable expression of the CD64 high-affinity Fc receptor over several weeks in culture.

### Example 5: NK Maturation on Control NK Cells and CD64 Transduced NK Cells is Maintained Over Time

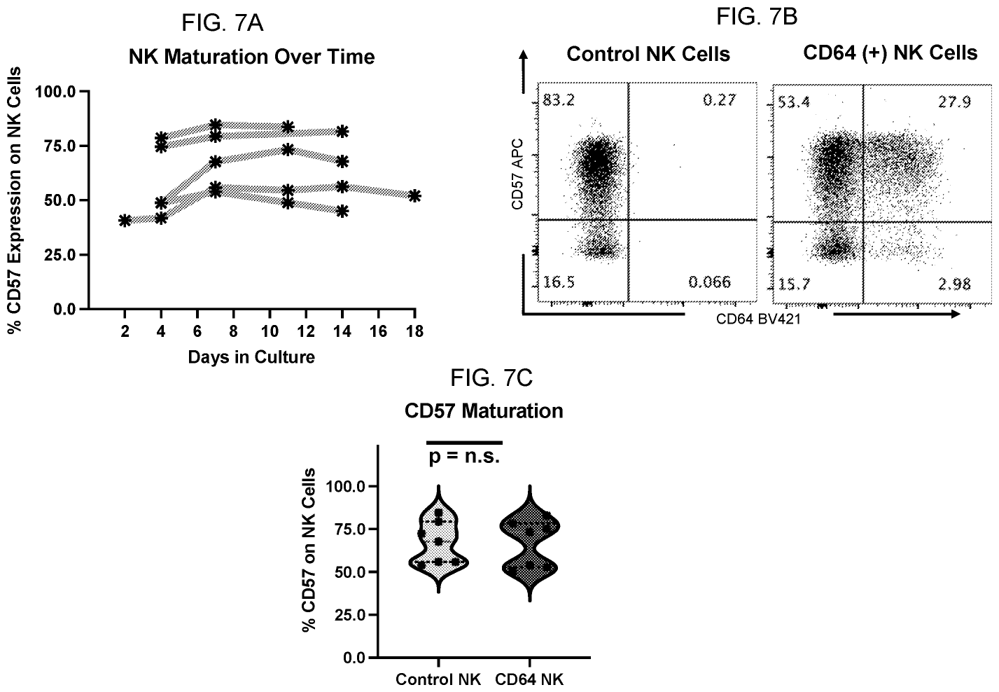

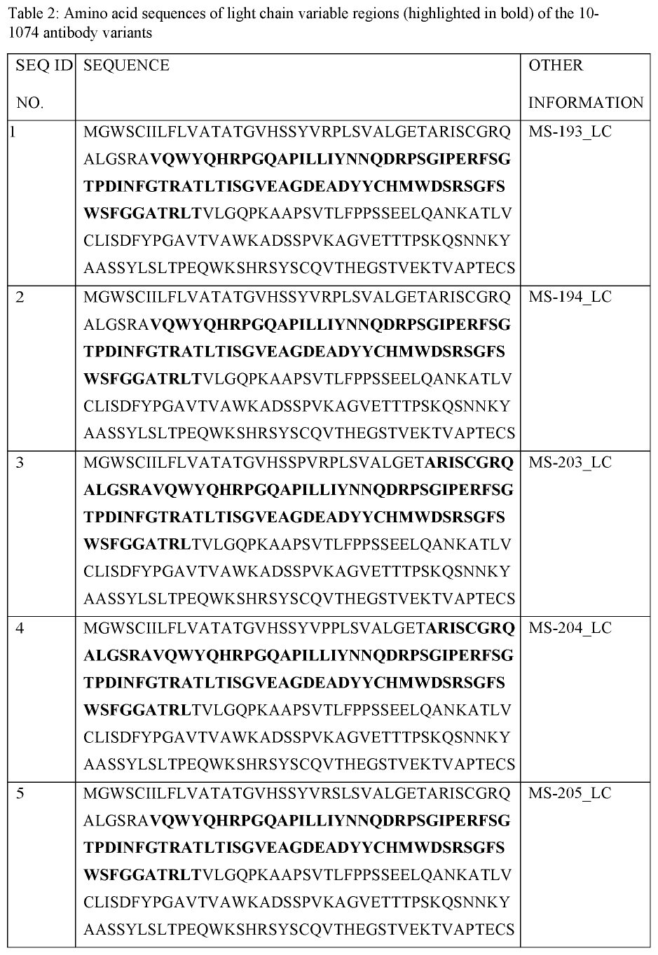

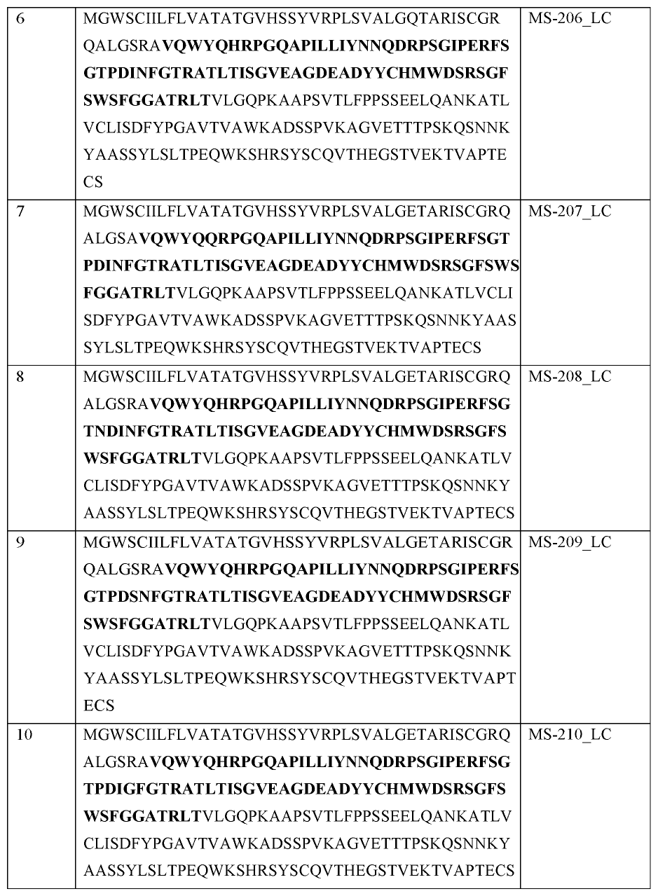

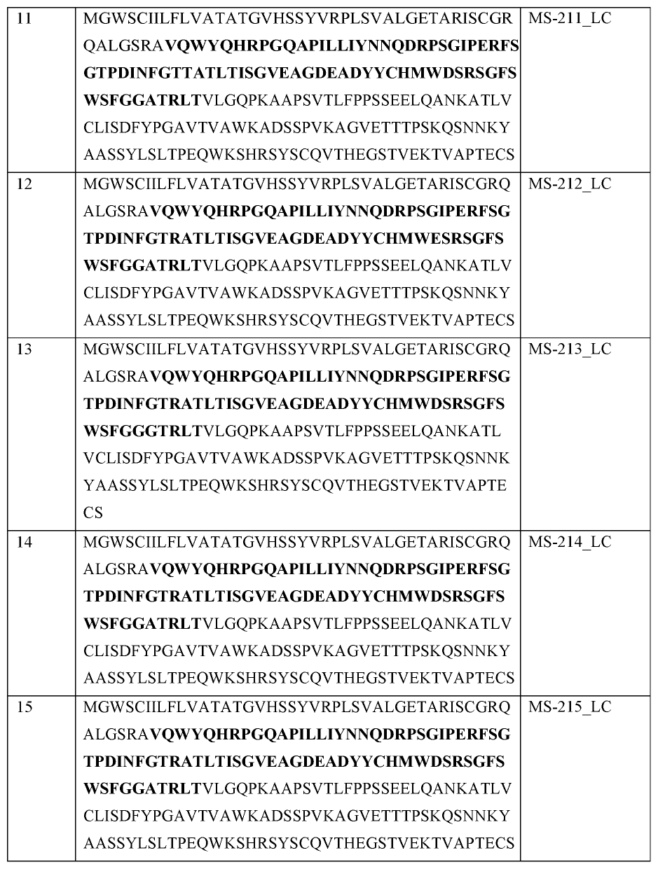

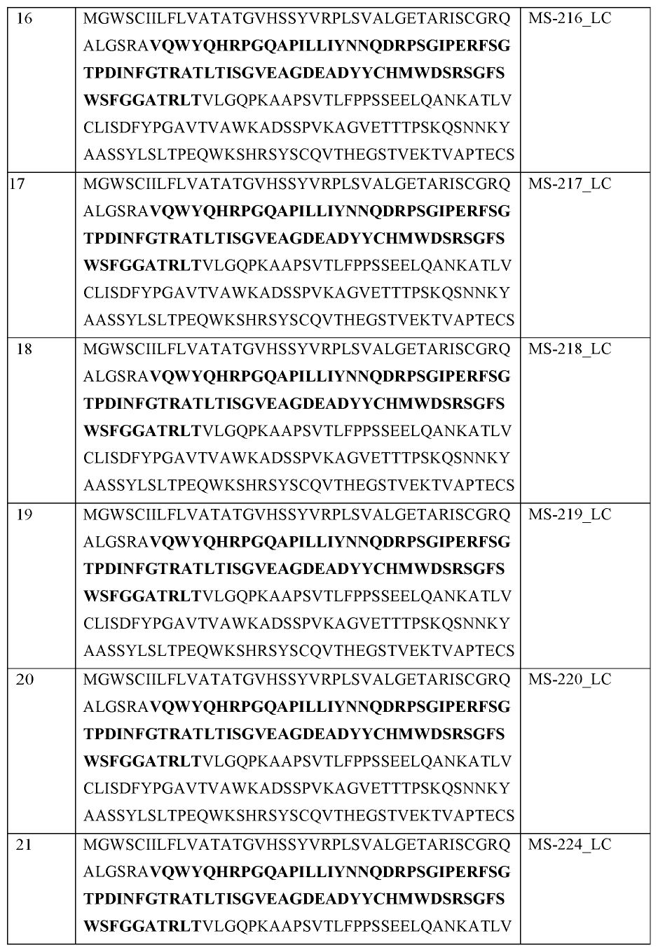

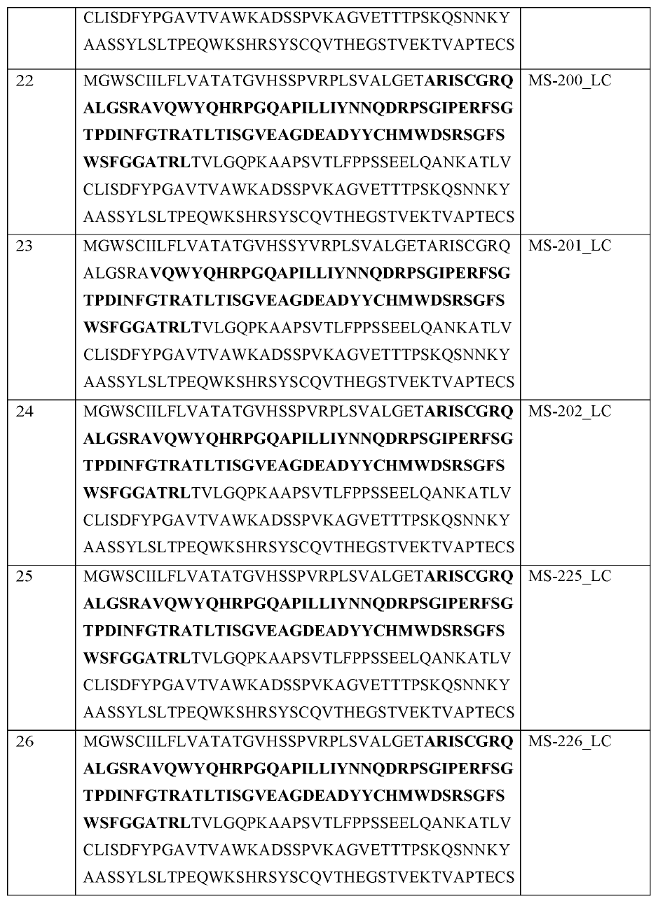

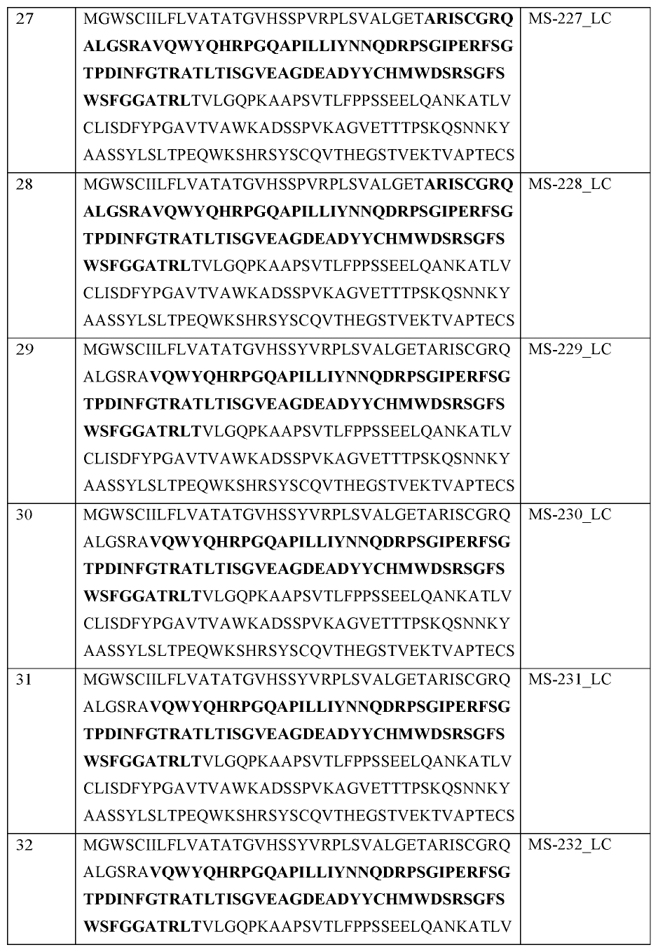

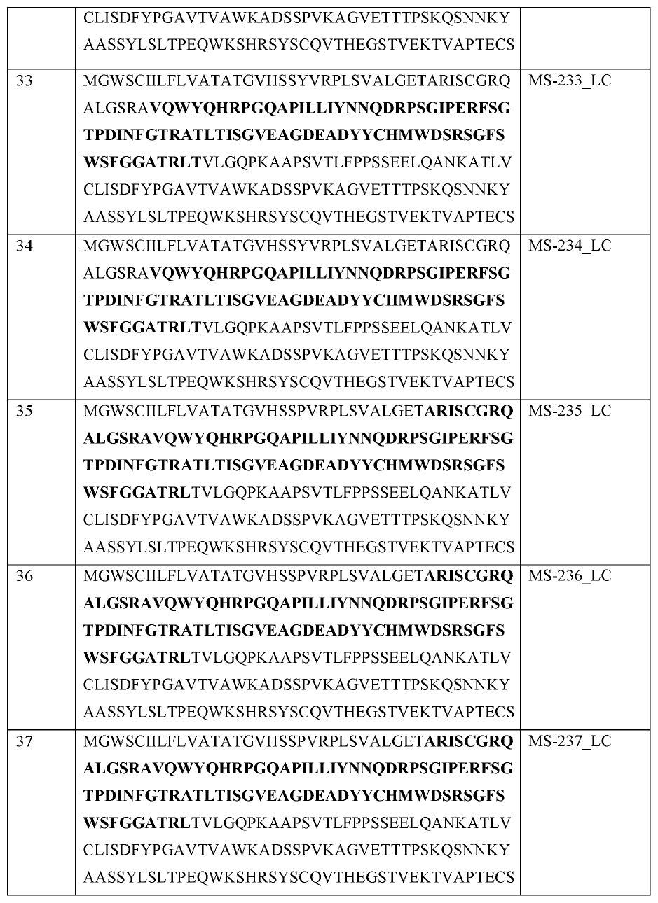

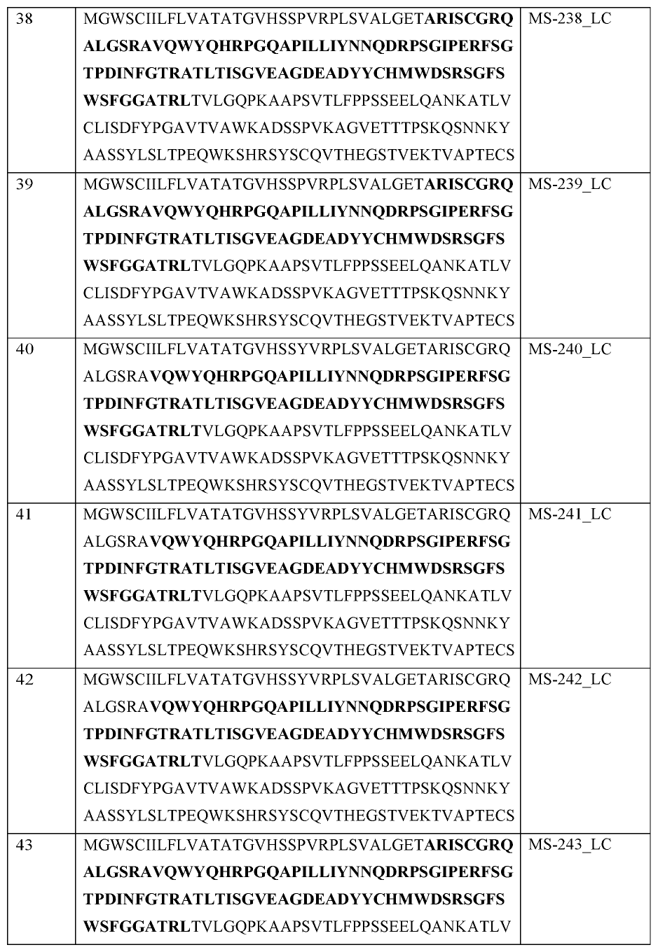

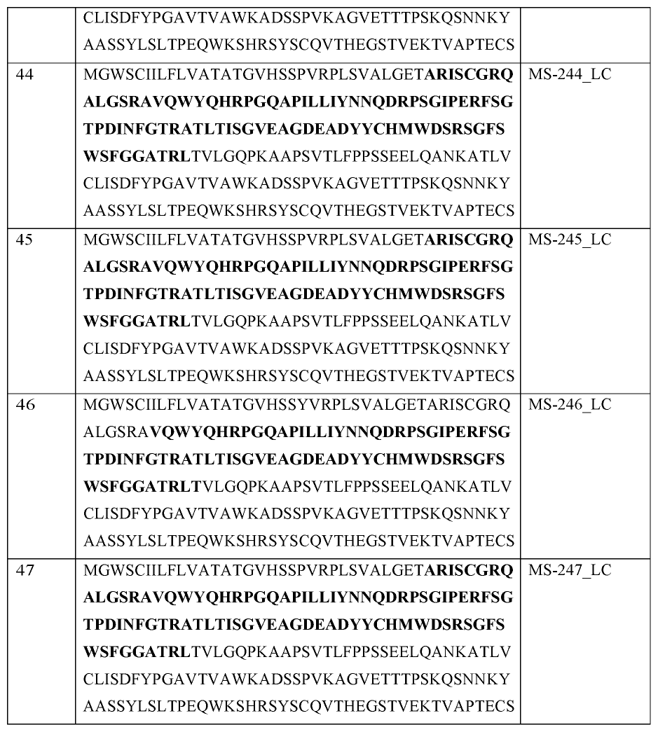

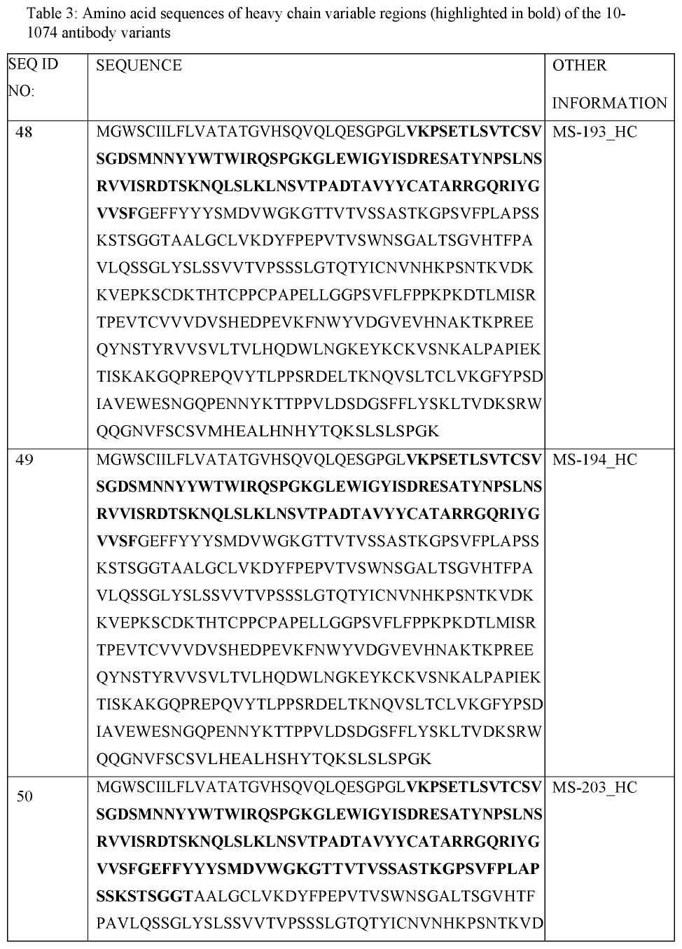

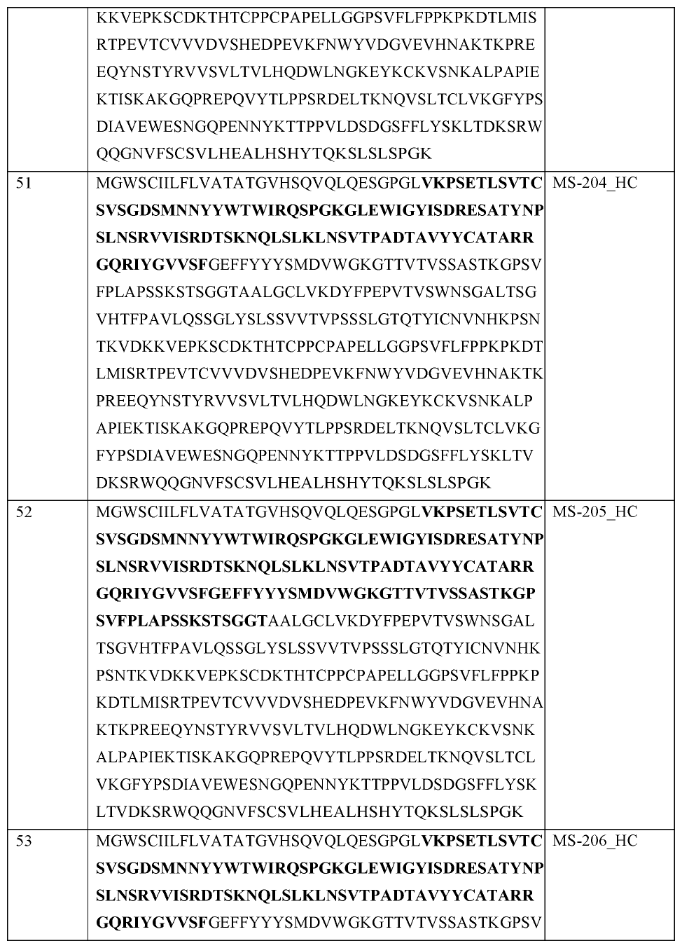

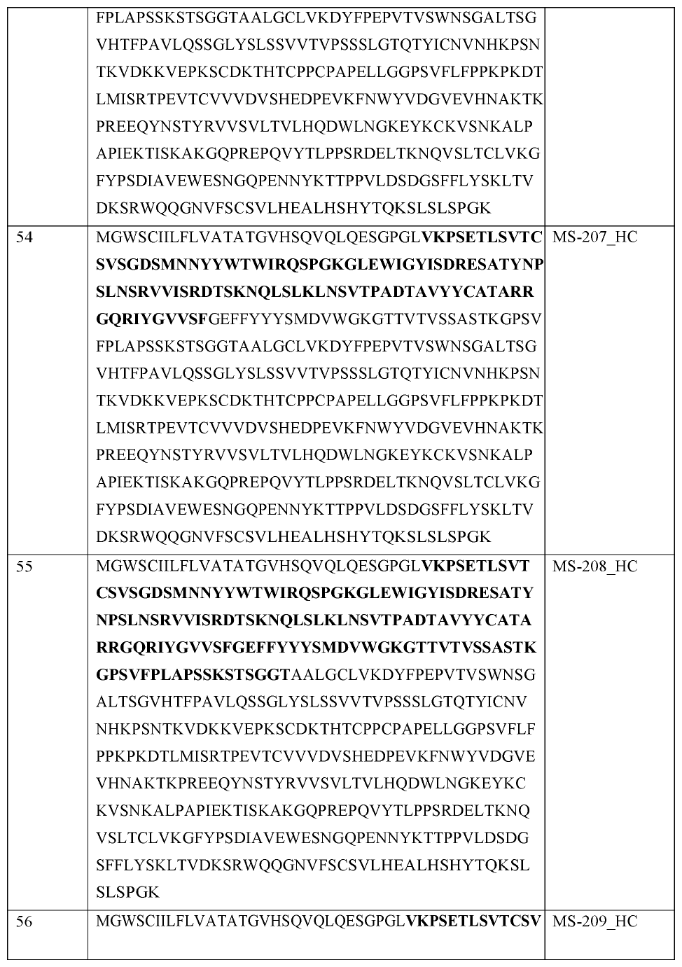

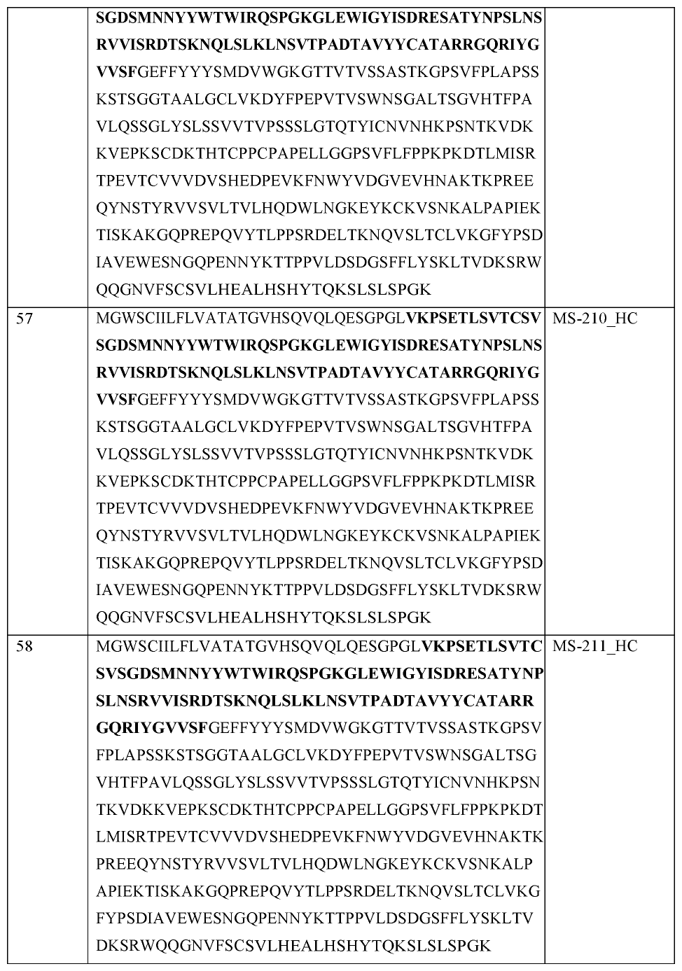

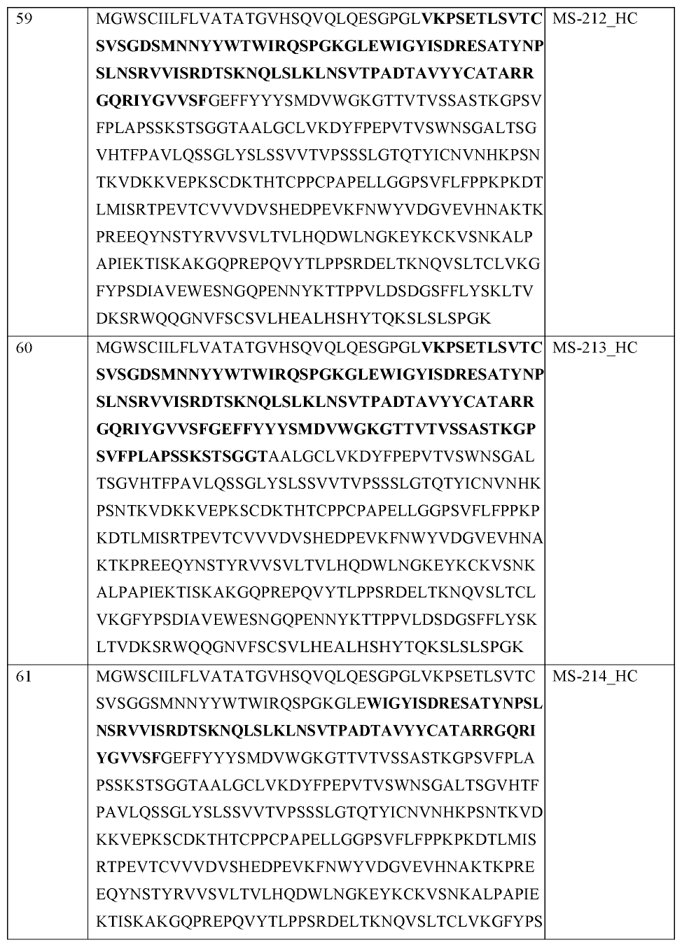

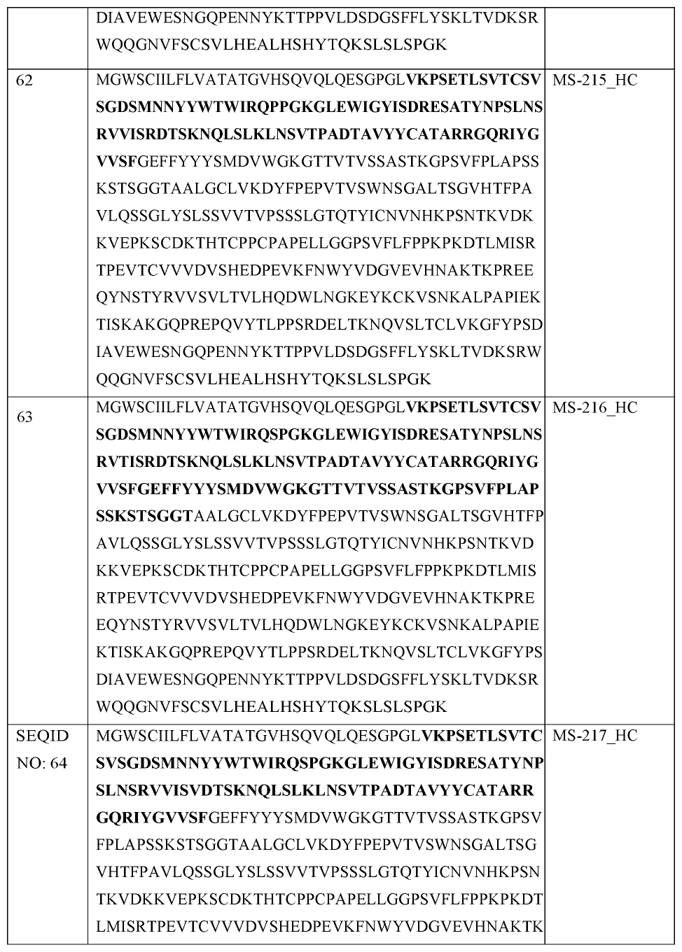

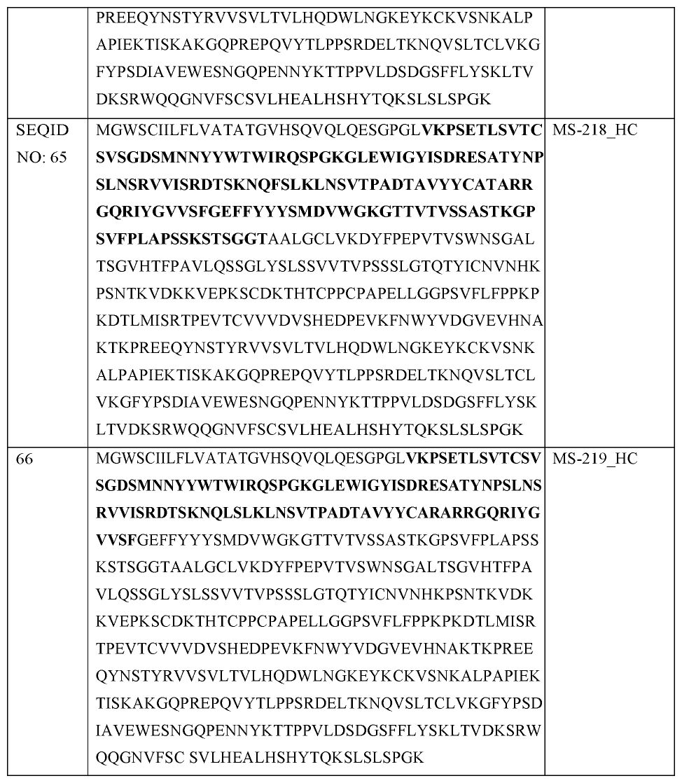

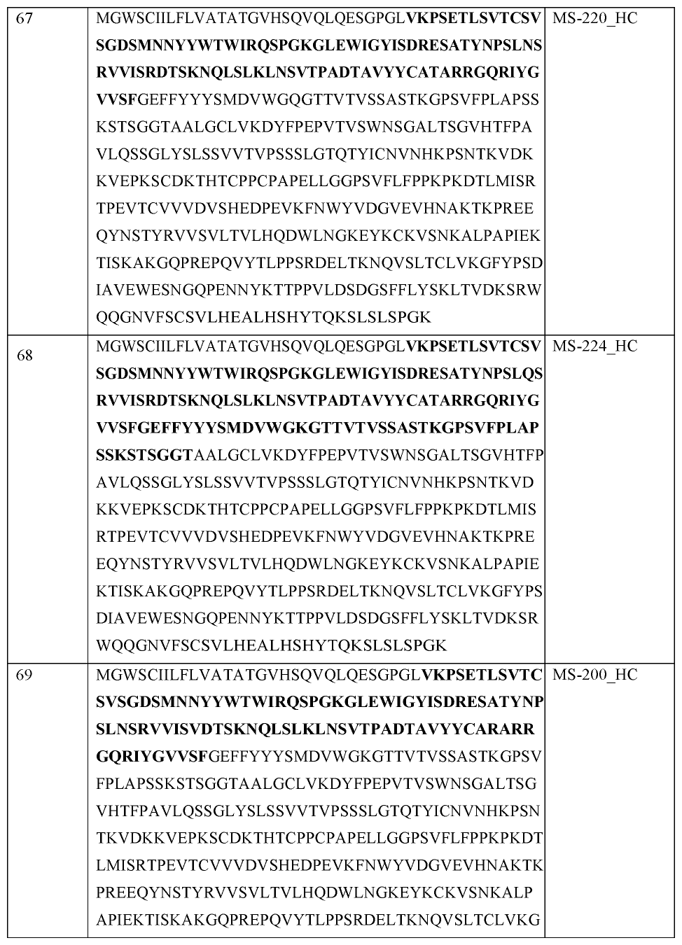

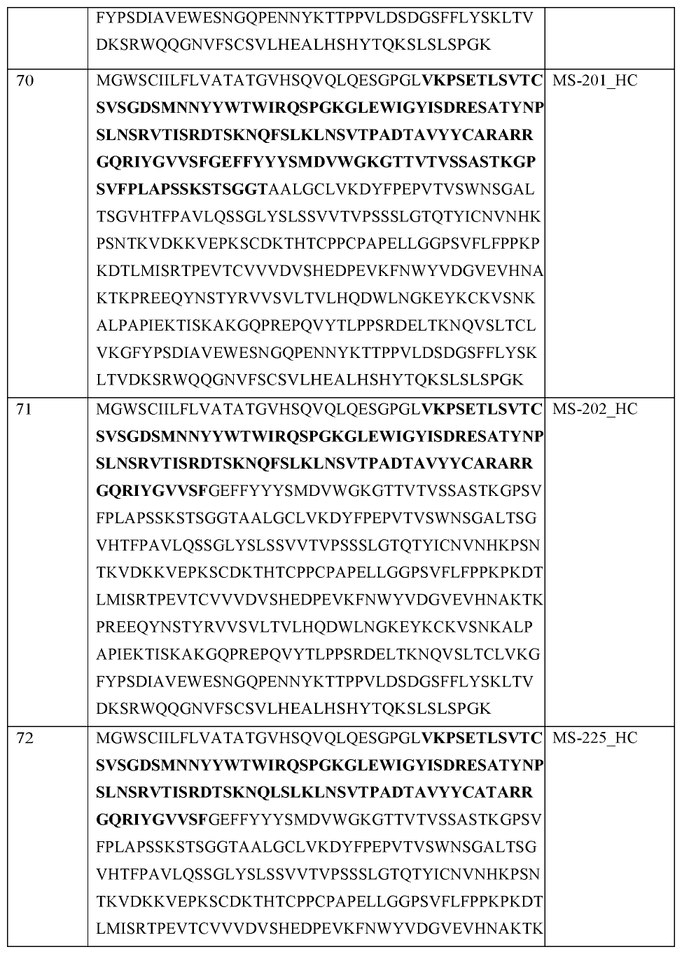

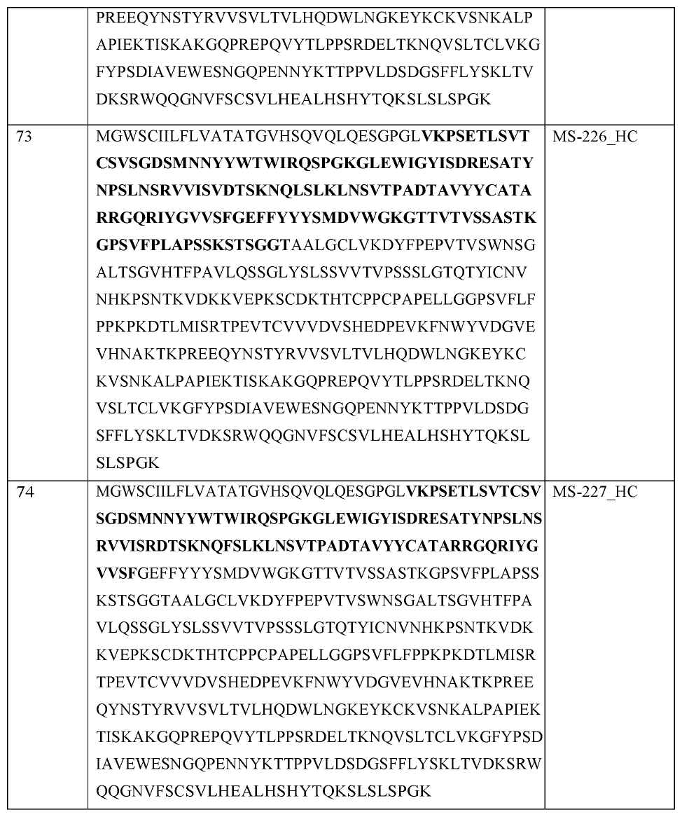

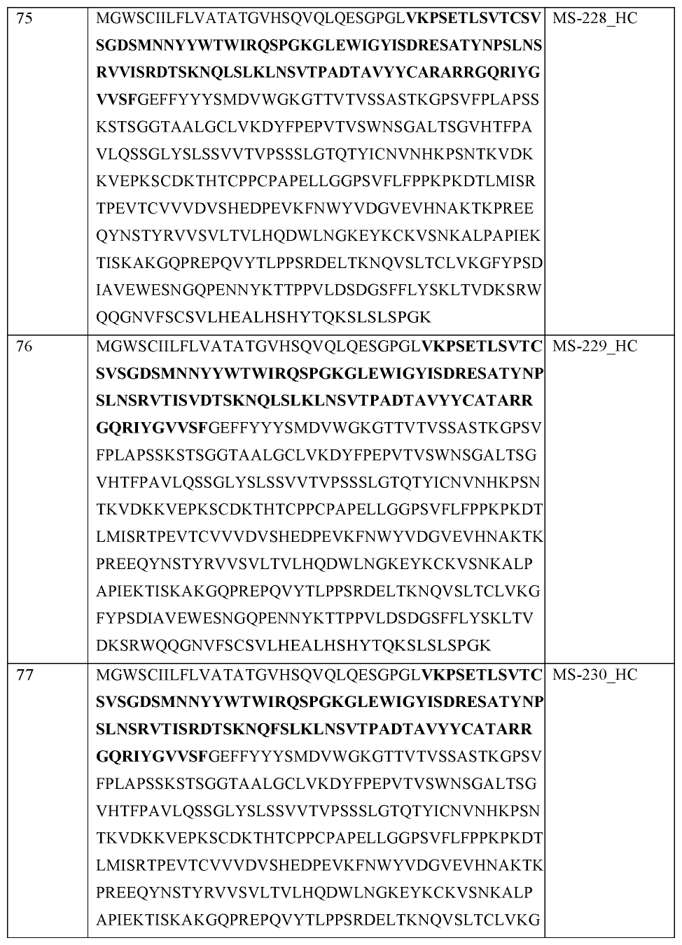

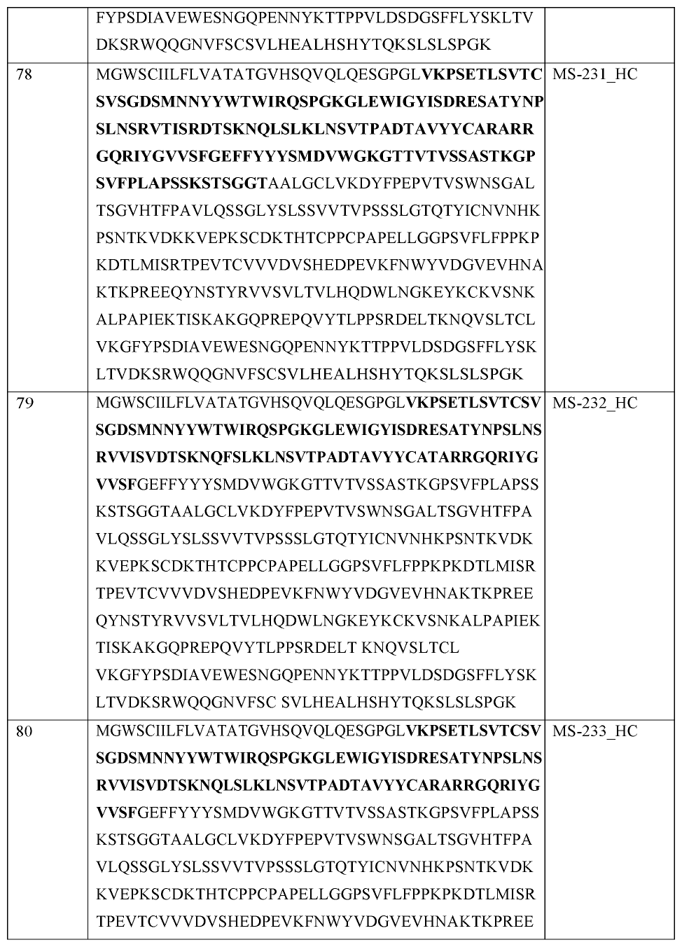

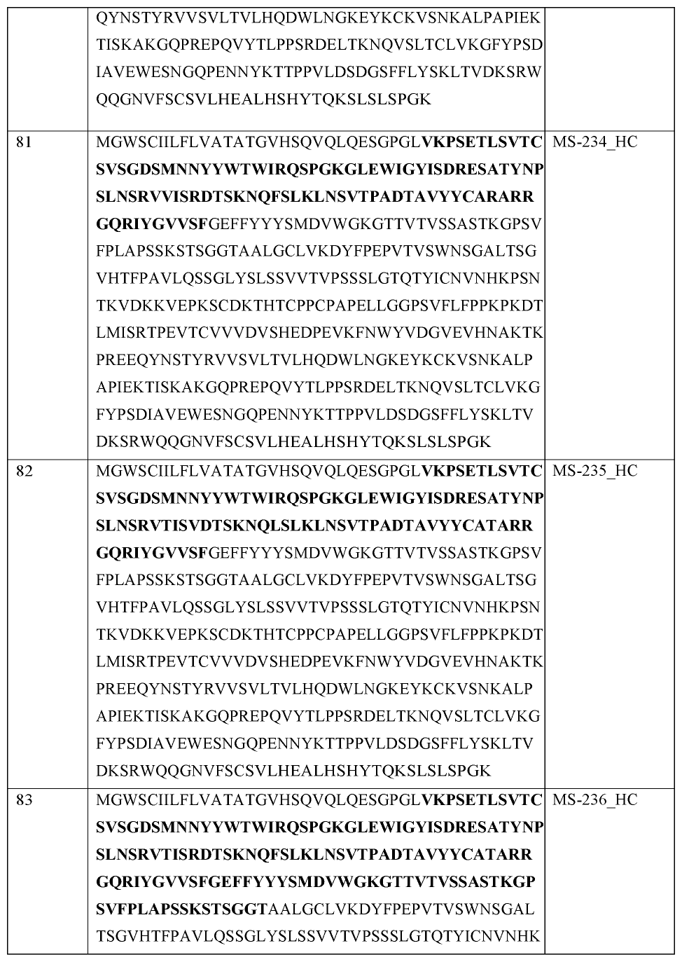

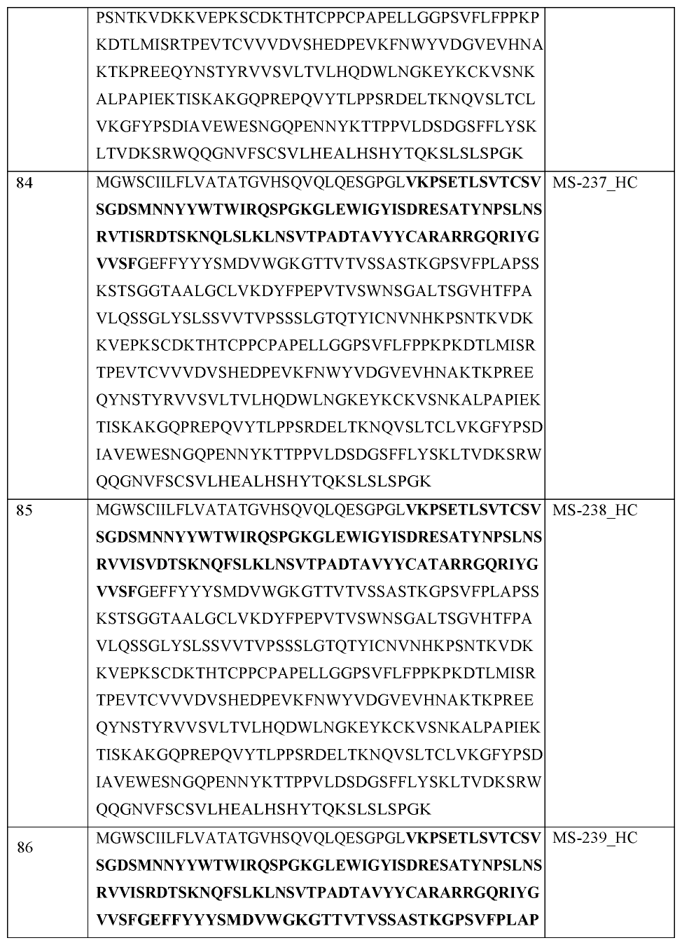

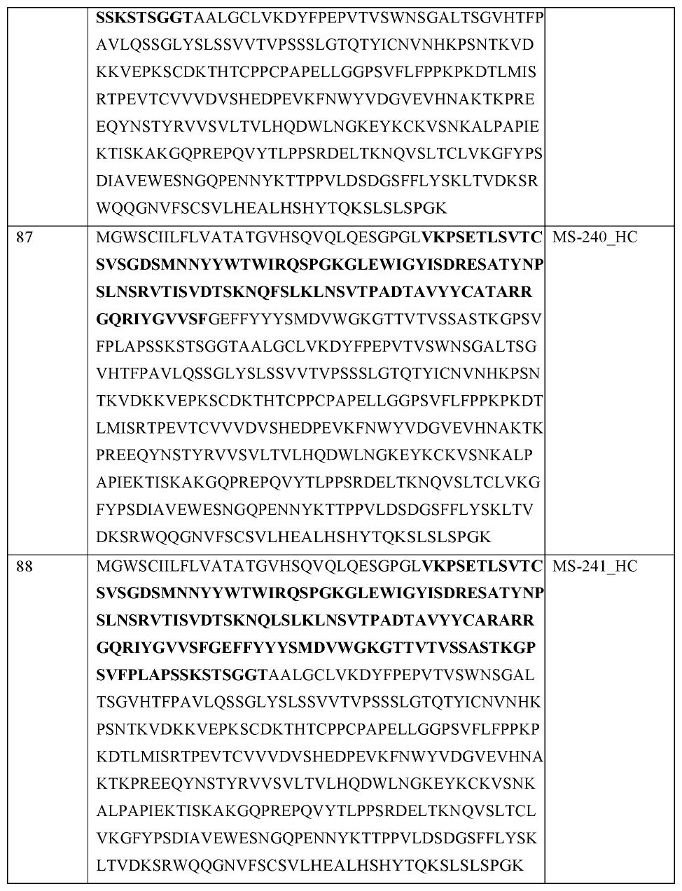

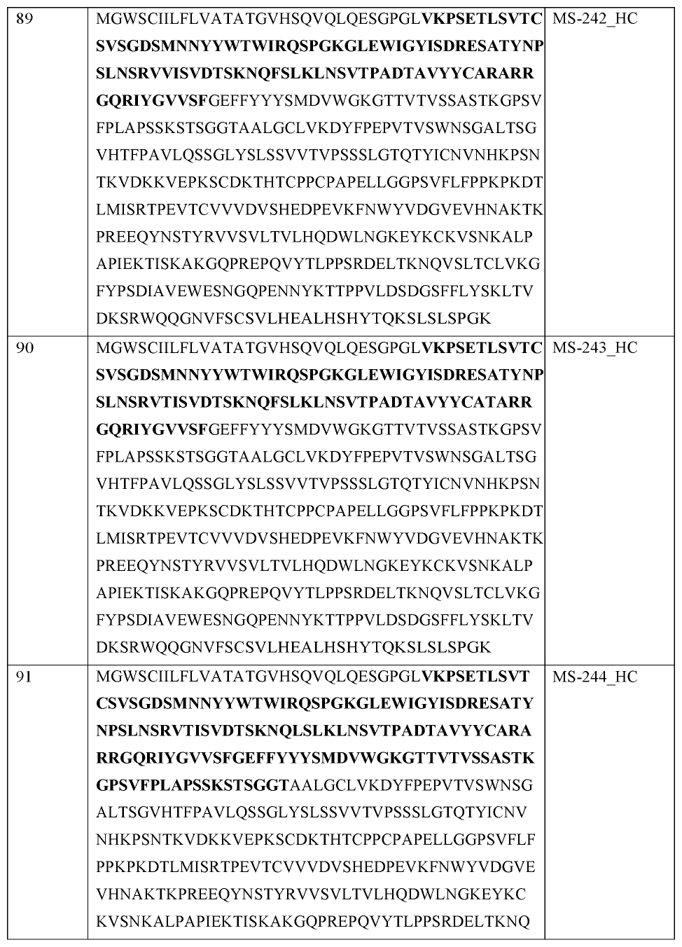

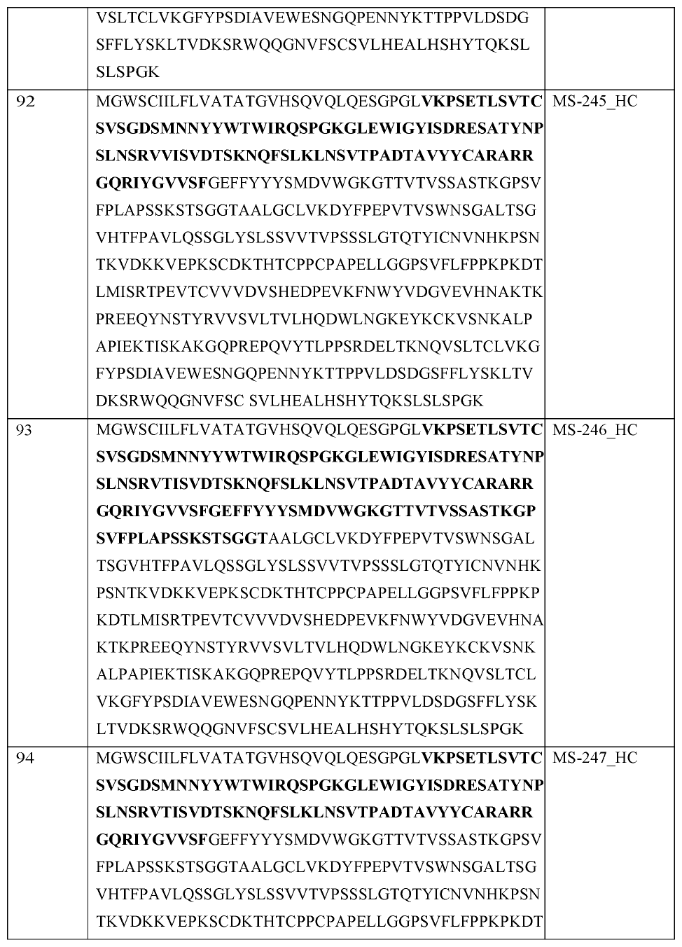

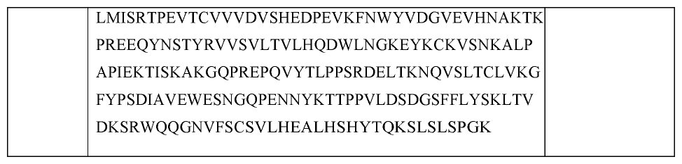

Human NK cells were purified by negative selection and stimulated with cytokines to maintain viability and NK maturation. Flow cytometry was used to monitor the expression of the CD57 maturation marker on NK cells over time (FIG. 7A). Following transduction with a Lentivirus encoding the high-affinity Fc receptor CD64, NK cell expression of the CD57 maturation marker was measured on CD64 transduced NK cells as well as control NK cells after 7 days (FIG. 7B).

### Results

Composite data shows CD57 expression remained was maintained and remained unaltered on transduced NK cells compared to control NK cells at Day 7 (FIG. 7C). This shows the ability of the cytokine cocktail and the protocol described in the instant invention to maintain CD57 maturation status in CD64 transduced NK cells over several weeks in culture. Maintenance of CD57 maturation status is associated with enhanced ADCC activity.

Each and every patent, patent application and any document identified herein and the sequence of any publicly available nucleic acid and/or peptide sequence cited throughout the disclosure is expressly incorporated herein by reference in its entirety. In addition, U.S. Provisional Patent Application No. 63/308,008, filed Feb. 8, 2022, is incorporated herein by reference in its entirety. Embodiments and variations of this invention other than those specifically disclosed above may be devised by others skilled in the art without departing from the true spirit and scope of the invention. The appended claims include such embodiments and equivalent variations.

### REFERENCES

Hintz H M, Snyder K M, Wu J, Hullsiek R, Dahlvang J D, Hart G T, Walcheck B, LeBeau A M. Simultaneous Engagement of Tumor and Stroma Targeting Antibodies by Engineered NK-92 Cells Expressing CD64 Controls Prostate Cancer Growth. Cancer Immunol Res. 2021 November; 9(11):1270-1282. doi: 10.1158/2326-6066.CIR-21-0178. Epub 2021 Aug. 27. PMID: 34452926.
Chen Y, You F, Jiang L, Li J, Zhu X, Bao Y, Sun X, Tang X, Meng H, An G, Zhang B, Yang L. Gene-modified NK-92MI cells expressing a chimeric CD16-BB-ζ or CD64-BB-ζ receptor exhibit enhanced cancer-killing ability in combination with therapeutic antibody. Oncotarget. 2017 Jun. 6; 8(23):37128-37139. doi: 10.18632/oncotarget.16201. PMID: 28415754; PMCID: PMC5514896.
Snyder K M, Hullsiek R, Mishra H K, Mendez D C, Li Y, Rogich A, Kaufman D S, Wu J, Walcheck B. Expression of a Recombinant High Affinity IgG Fc Receptor by Engineered NK Cells as a Docking Platform for Therapeutic mAbs to Target Cancer Cells. Front Immunol. 2018 Dec. 6; 9:2873. doi: 10.3389/fimmu.2018.02873. PMID: 30574146; PMCID: PMC6291448.
US 2020/0283501 (Wu et al), published Sep. 10, 2020.

1. A composition comprising primary human NK cells that express CD64 Fc receptor from an exogenous nucleic acid molecule, the NK cells having bound thereto anti-HIV antibodies.

2. The composition according to claim 1, wherein the anti-HIV antibodies are broadly neutralizing antibodies.

3. The composition according to claim 1 or 2, wherein the anti-HIV antibodies are 10-1074 antibodies, or variants thereof.

4. The composition according to any one of claims 1 or 2, wherein the anti-HIV antibodies are 10-259, 10-303, 10-410, 10-847, 10-996, 10-1121, 10-1130, 10-1146, 10-1341, 10-1369, 10-1074GM, GL, 10E8, 12A12, 12A21, 2F5, 2G12, 35022, 3BC176, 3BNC117, 3BNC55, 3BNC60, 3BNC62, 447-52D, 4E10, 5H/I1-BMV-D5, 8ANC195, b12, CAP256-VRC26.01, CAP256-VRC26.02, CAP256-VRC26.03, CAP256-VRC26.04, CAP256-VRC26.05, CAP256-VRC26.06, CAP256-VRC26.07, CAP256-VRC26.08, CAP256-VRC26.09, CAP256-VRC26.10, CAP256-VRC26.il, CAP256-VRC26.12, CHOI, CH02, CH03, CHO4, CH103, HGN194, HJ16, HK20, M66.6, NIH45-46, PCDN-33A, PCDN-33B, PCDN-38A, PG9, PG16, PGDM1400, PGDM1401, PGDM1402, PGDM1403, PGDM1404, PGDM1405, PGDM1406, PGDM1407, PGDM1408, PGDM1409, PGDM1410, PGDM1411, PGDM1412, PGT121, PGT122, PGT123, PGT125, PGT126, PGT127, PGT128, PGT130, PGT131, PGT135, PGT136, PGT137, PGT141, PGT142, PGT143, PGT145, PGT151, PGT152, VRC-CH30, VRC-CH31, VRC-CH32, VRC-CH33, VRC-CH34, VRC-PG04, VRC-CHO4b, VRC-PG20, VRCO1, VRC02, VRC03, VRC07, VRC23, or Z13.

5. The composition according to claim 4, wherein the anti-HIV antibody is 3BNC117.

6. The composition according to any one of claims 1 to 5, wherein the CD64 Fc receptor is a native human CD64 Fc receptor.

7. A method of inducing or augmenting antibody-dependent cellular cytotoxicity (ADCC) in a subject in need thereof, the method comprising administering a composition comprising human NK cells that express high affinity CD64 Fc receptor from an exogenous nucleic acid molecule, the NK cells having antibodies bound thereto.

8. The method according to claim 7, wherein the human NK cells are removed from the subject and transduced ex vivo with the exogenous nucleic acid molecule that encodes CD64 prior to administering to the subject.

9. The method according to claim 7 or 8, wherein the transduced NK cells are cultured in media supplemented with one or more cytokines.

10. The method according to claim 9, wherein the transduced NK cells are cultured for at least 7 days.

11. The method according to any one of claims 9-10, wherein the NK cells are contacted with IL-21.

12. The method according to any one of claims 7 to 11, wherein the antibodies are anti-HIV antibodies.

13. The method according to any one of claims 7 to 11, wherein the antibodies are anti-SARS-COV-2 antibodies.

14. The method according to any one of claims 7 to 11, wherein the antibodies are anti-cancer antibodies.

15. The method according to any one of claims 7 to 11, wherein the antibodies are antibodies against a viral or bacterial pathogen.

16. A composition comprising primary human NK cells that express CD64 Fc receptor from an exogenous nucleic acid molecule, the NK cells having antibodies bound thereto.

17. The composition according to claim 7, wherein the antibodies are anti-cancer antibodies.

18. The composition according to claim 7, wherein the antibodies are antibodies against a viral or bacterial pathogen.

19. A method of generating modified human NK cells, the method comprising removing NK cells from a subject and transducing the cells ex vivo with an exogenous nucleic acid molecule that encodes CD64, contacting the NK cells with an antibody, thereby generating modified NK cells having antibodies bound thereto.

20. The method according to claim 19, wherein the transduced NK cells are cultured in media supplemented with one or more cytokines.

21. The method according to claim 20, wherein the transduced NK cells are cultured for at least 7 days.

22. The method according to claim 20, wherein the transduced NK cells are cultured for with IL-21 for the entire culturing period.

23. Use of the composition according to any one of claims 1 to 6, for treatment of HIV-1 in a subject in need thereof.

24. Use of the composition according to any one of claims 16 to 18, for treatment of cancer in a subject in need thereof.

25. Use of the composition according to any one of claims 16 to 18, for treatment of a bacterial or viral infection in a subject in need thereof.


SEQUENCE LISTING (96 sequences)

SEQ ID NO: 1 - protein, 233 aa, synthetic construct
MGWSCIILFLVATATGVHSSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 2 - protein, 233 aa, synthetic construct; identical to SEQ ID NO: 1

SEQ ID NO: 3 - protein, 233 aa, synthetic construct
MGWSCIILFLVATATGVHSSPVRPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 4 - protein, 233 aa, synthetic construct
MGWSCIILFLVATATGVHSSYVPPLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 233 aa, synthetic construct
MGWSCIILFLVATATGVHSSYVRSLSVALGETARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - protein, 233 aa, synthetic construct
MGWSCIILFLVATATGVHSSYVRPLSVALGQTARISCGRQALGSRAVQWYQHRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 232 aa, synthetic construct
MGWSCIILFLVATATGVHSSYVRPLSVALGETARISCGRQALGSAVQWYQQRPGQAPILLIYNNQDRPSGIPERFSGTPDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - protein, 233 aa, synthetic construct
MGWSCIILFLVATATGVHSSYVRPLSVALGETARISCGRQALGSRAVQWYQHRPGQAPILLIYNNQDRPSGIPERFSGTNDINFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 9 - protein, 233 aa, synthetic construct
MGWSCIILFLVATATGVHSSYVRPLSVALGETARISCGRQALGSRAVQWYQHRPGQAPILLIYNNQDRPSGIPERFSGTPDSNFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 233 aa, synthetic construct
MGWSCIILFLVATATGVHSSYVRPLSVALGETARISCGRQALGSRAVQWYQHRPGQAPILLIYNNQDRPSGIPERFSGTPDIGFGTRATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 233 aa, synthetic construct
MGWSCIILFLVATATGVHSSYVRPLSVALGETARISCGRQALGSRAVQWYQHRPGQAPILLIYNNQDRPSGIPERFSGTPDINFGTTATLTISGVEAGDEADYYCHMWDSRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 233 aa, synthetic construct
MGWSCIILFLVATATGVHSSYVRPLSVALGETARISCGRQALGSRAVQWYQHRPGQAPILLIYNNQDRPSGIPERFSGTPDINFGTRATLTISGVEAGDEADYYCHMWESRSGFSWSFGGATRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 233 aa, synthetic construct
MGWSCIILFLVATATGVHSSYVRPLSVALGETARISCGRQALGSRAVQWYQHRPGQAPILLIYNNQDRPSGIPERFSGTPDINFGTRATLTISGVEAGDEADYYCHMWDSRSGFSWSFGGGTRLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 14 - protein, 233 aa, synthetic construct; identical to SEQ ID NO: 1

SEQ ID NO: 15 - protein, 233 aa, synthetic construct; identical to SEQ ID NO: 1

SEQ ID NO: 16 - protein, 233 aa, synthetic construct; identical to SEQ ID NO: 1

SEQ ID NO: 17 - protein, 233 aa, synthetic construct; identical to SEQ ID NO: 1

SEQ ID NO: 18 - protein, 233 aa, synthetic construct; identical to SEQ ID NO: 1

SEQ ID NO: 19 - protein, 233 aa, synthetic construct; identical to SEQ ID NO: 1

SEQ ID NO: 20 - protein, 233 aa, synthetic construct; identical to SEQ ID NO: 1

SEQ ID NO: 21 - protein, 233 aa, synthetic construct; identical to SEQ ID NO: 1

SEQ ID NO: 22 - protein, 233 aa, synthetic construct; identical to SEQ ID NO: 3

SEQ ID NO: 23 - protein, 233 aa, synthetic construct; identical to SEQ ID NO: 1

SEQ ID NO: 24 - protein, 233 aa, synthetic construct; identical to SEQ ID NO: 3

SEQ ID NO: 25 - protein, 233 aa, synthetic construct; identical to SEQ ID NO: 3

SEQ ID NO: 26 - protein, 233 aa, synthetic construct; identical to SEQ ID NO: 3

SEQ ID NO: 27 - protein, 233 aa, synthetic construct; identical to SEQ ID NO: 3

SEQ ID NO: 28 - protein, 233 aa, synthetic construct; identical to SEQ ID NO: 3

SEQ ID NO: 29 - protein, 233 aa, synthetic construct; identical to SEQ ID NO: 1

SEQ ID NO: 30 - protein, 233 aa, synthetic construct; identical to SEQ ID NO: 1

SEQ ID NO: 31 - protein, 233 aa, synthetic construct; identical to SEQ ID NO: 1

SEQ ID NO: 32 - protein, 233 aa, synthetic construct; identical to SEQ ID NO: 1

SEQ ID NO: 33 - protein, 233 aa, synthetic construct; identical to SEQ ID NO: 1

SEQ ID NO: 34 - protein, 233 aa, synthetic construct; identical to SEQ ID NO: 1

SEQ ID NO: 35 - protein, 233 aa, synthetic construct; identical to SEQ ID NO: 3

SEQ ID NO: 36 - protein, 233 aa, synthetic construct; identical to SEQ ID NO: 3

SEQ ID NO: 37 - protein, 233 aa, synthetic construct; identical to SEQ ID NO: 3

SEQ ID NO: 38 - protein, 233 aa, synthetic construct; identical to SEQ ID NO: 3

SEQ ID NO: 39 - protein, 233 aa, synthetic construct; identical to SEQ ID NO: 3

SEQ ID NO: 40 - protein, 233 aa, synthetic construct; identical to SEQ ID NO: 1

SEQ ID NO: 41 - protein, 233 aa, synthetic construct; identical to SEQ ID NO: 1

SEQ ID NO: 42 - protein, 233 aa, synthetic construct; identical to SEQ ID NO: 1

SEQ ID NO: 43 - protein, 233 aa, synthetic construct; identical to SEQ ID NO: 3

SEQ ID NO: 44 - protein, 233 aa, synthetic construct; identical to SEQ ID NO: 3

SEQ ID NO: 45 - protein, 233 aa, synthetic construct; identical to SEQ ID NO: 3

SEQ ID NO: 46 - protein, 233 aa, synthetic construct; identical to SEQ ID NO: 1

SEQ ID NO: 47 - protein, 233 aa, synthetic construct; identical to SEQ ID NO: 3

SEQ ID NO: 48 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 50 - protein, 480 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTDKSRWQQGNVFSCSVLHEALHSHYTQKSLSLSPGK

SEQ ID NO: 51 - protein, 481 aa, synthetic construct; identical to SEQ ID NO: 49

SEQ ID NO: 52 - protein, 481 aa, synthetic construct; identical to SEQ ID NO: 49

SEQ ID NO: 53 - protein, 481 aa, synthetic construct; identical to SEQ ID NO: 49

SEQ ID NO: 54 - protein, 481 aa, synthetic construct; identical to SEQ ID NO: 49

SEQ ID NO: 55 - protein, 481 aa, synthetic construct; identical to SEQ ID NO: 49

SEQ ID NO: 56 - protein, 481 aa, synthetic construct; identical to SEQ ID NO: 49

SEQ ID NO: 57 - protein, 481 aa, synthetic construct; identical to SEQ ID NO: 49

SEQ ID NO: 58 - protein, 481 aa, synthetic construct; identical to SEQ ID NO: 49

SEQ ID NO: 59 - protein, 481 aa, synthetic construct; identical to SEQ ID NO: 49

SEQ ID NO: 60 - protein, 481 aa, synthetic construct; identical to SEQ ID NO: 49

SEQ ID NO: 61 - protein, 481 aa, synthetic construct
MGWSCIILFLVATATGVHSQVQLQESGPGLVKPSETLSVTCSVSGGSMNNYYWTWIRQSPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 62 - protein, 481 aa, synthetic construct
MGWSCIILFLVATATGVHSQVQLQESGPGLVKPSETLSVTCSVSGDSMNNYYWTWIRQPPGKGLEWIGYISDRESATYNPSLNSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 63 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 64 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISVDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 65 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQFSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 66 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQLSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 67 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQLSLKLNSVTPADTAVYYCATARRGQRIYGVVSFGEFFYYYSMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 68 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLQSRVVISRDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 69 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISVDTSKNQLSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 70 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISRDTSKNQFSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 71 - protein, 481 aa, synthetic construct; identical to SEQ ID NO: 70

SEQ ID NO: 72 - protein, 481 aa, synthetic construct; identical to SEQ ID NO: 63

SEQ ID NO: 73 - protein, 481 aa, synthetic construct; identical to SEQ ID NO: 64

SEQ ID NO: 74 - protein, 481 aa, synthetic construct; identical to SEQ ID NO: 65

SEQ ID NO: 75 - protein, 481 aa, synthetic construct; identical to SEQ ID NO: 66

SEQ ID NO: 76 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISVDTSKNQLSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 77 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISRDTSKNQFSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 78 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISRDTSKNQLSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 79 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISVDTSKNQFSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 80 - protein, 481 aa, synthetic construct; identical to SEQ ID NO: 69

SEQ ID NO: 81 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQFSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 82 - protein, 481 aa, synthetic construct; identical to SEQ ID NO: 76

SEQ ID NO: 83 - protein, 481 aa, synthetic construct; identical to SEQ ID NO: 77

SEQ ID NO: 84 - protein, 481 aa, synthetic construct; identical to SEQ ID NO: 78

SEQ ID NO: 85 - protein, 481 aa, synthetic construct; identical to SEQ ID NO: 79

SEQ ID NO: 86 - protein, 328 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISRDTSKNQFSLKLNSVTPADTAVYYCARARRGQRIYGVVSFGEFFYYYSMDVWGKGTTVTVSSASTKGPSVFPLAPMGWSCIILFLVATATGVHSQVQLQESGPGLVKPSETLSVTCSVSGDSMNNYYWTWIRQSPGKGLEWIGYISDRESATYNPSLNSRVVISRDTSKNQFSLKLNSVTPADTAVYYCARARRGQRIYGVVSFGEFFYYYSMDVWGKGTTVTVSSASTKGPSVFPLAP

SEQ ID NO: 87 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISVDTSKNQFSLKLNSVTPADTAVYYCATARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 88 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISVDTSKNQLSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 89 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVVISVDTSKNQFSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 90 - protein, 481 aa, synthetic construct; identical to SEQ ID NO: 89

SEQ ID NO: 91 - protein, 792 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISVDTSKNQLSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQMGWSCIILFLVATATGVHSQVQLQESGPGLVKPSETLSVTCSVSGDSMNNYYWTWIRQSPGKGLEWIGYISDRESATYNPSLNSRVTISVDTSKNQLSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQ

SEQ ID NO: 92 - protein, 481 aa, synthetic construct; identical to SEQ ID NO: 89

SEQ ID NO: 93 - protein, 481 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISVDTSKNQFSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 94 - protein, 568 aa, synthetic construct
MGWSCIILFLVATATGVHSQVQLQESGPGLVKPSETLSVTCSVSGDSMNNYYWTWIRQSPGKGLEWIGYISDRESATYNPSLNSRVTISVDTSKNQFSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTMGWSCIILFLVATATGVHSQVQLQESGPGLVKPSETLSVTCSVSGDSMNNYYWTWIRQSPGKGLEWIGYISDRESATYNPSLNSRVTISVDTSKNQFSLKLNSVTPADTAVYYCARARRGQRIYGVVSFGEFFYYYSMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDT

SEQ ID NO: 95 - protein, 280 aa, Homo sapiens
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFMEGEPLALRCHAWKDKLVYNVLYYRNGKAFKFFHWNSNLTILKTNISHNGTYHCSGMGKHRYTSAGISQYTVKGLQLPTPVWFHVLFYLAVGIMFLVNTVLWVTIRKELKRKKKWNLEISLDSGHEKKVISSLQEDRHLEEELKCQEQKEEQLQEGVHRKEPQGAT

SEQ ID NO: 96 - DNA, 904 bp, Homo sapiens
  source 1..904: mol_type = other DNA
gtacaaaaaagcagaagggccgtcaaggcccaccatgtggttcttgacaactctgctcctttgggttccagttgatgggcaagtggacaccacaaaggcagtgatcactttgcagcctccatgggtcagcgtgttccaagaggaaaccgtaaccttgcactgtgaggtgctccatctgcctgggagcagctccacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaaggcctccagttaccaactcctgtctggtttcatgtccttttctatctggcagtgggaataatgtttttagtgaacactgttctctgggtgacaatacgtaaagaactgaaaagaaagaaaaagtggaatttagaaatctctttggattctggtcatgagaagaaggtaatttccagccttcaagaagacagacatttagaagaagagctgaaatgtcaggaacaaaaagaagaacagctgcaggaaggggtgcaccggaaggagccccagggggccacgtaaggcctcatgggcccagctttcttgtac